(19) United States
(12) Patent Application Publication
(10) Pub. No.: US 2025/0134929 A1
(43) Pub. Date: May 1, 2025

(54) ORTHOGONAL GAMMA CHAINS AND SYSTEMS COMPRISING THE SAME

(71) Applicant: Neptune Biosciences, LLC, Newark, CA (US)

(72) Inventor: Nigel Killeen, Hillsborough, CA (US)

(73) Assignee: Neptune Biosciences, LLC, Newark, CA (US)

(21) Appl. No.: 18/911,480

(22) Filed: Oct. 10, 2024

Related U.S. Application Data
(60) Provisional application No. 63/593,188, filed on Oct. 25, 2023.

(51) Int. Cl. A61K 35/17; A61K 40/11; A61K 40/31; A61K 40/42; C07K 14/715

(52) U.S. Cl. CPC A61K 35/17; A61K 40/11; A61K 40/31; A61K 40/4217; C07K 14/7155

(57) ABSTRACT

Variant IL2RG (CD132 or common-γ or γc) chains are provided that are capable of rendering cells responsive to variant cytokines.

### CROSS-REFERENCE TO RELATED APPLICATIONS

This application claims the benefit of U.S. Provisional Patent Application No. 63/593,188, filed on Oct. 25, 2023, which is incorporated by reference herein in its entirety.

### SEQUENCE LISTING

A Sequence Listing has been submitted electronically in XML format and is hereby incorporated by reference in its entirety. The XML copy, created on Oct. 25, 2023, is named OGC_ST26.xml and is 541,992 bytes in size.

### BACKGROUND

Cytokines are potent natural regulators of immune cell proliferation and differentiation. While this potency has made cytokines highly attractive as potential therapeutics, it has also complicated their clinical utility. This has been especially true for cytokines that have multiple cellular targets and, thus, high potential for pleiotropic effects. One example is Interleukin-2 (“IL-2”), a robust T cell mitogen whose anti-cancer activity is offset by unwanted proliferation of regulatory (suppressor) T cells and a painful vascular leak syndrome. In the specific case of IL-2, protein engineering can be used to solve some of the clinical challenges: removing, for example, the cytokine's capacity to act preferentially on regulatory T cells. An alternative approach involves generating orthogonally constrained forms of cytokines and their receptors. See U.S. patent application Ser. No. 18/304,172; U.S. Pat. No. 10,869,887; Sockolosky J T, Trotta E, Parisi G, et al. Selective targeting of engineered T cells using orthogonal IL-2 cytokine-receptor complexes. Science. 2018; 359 (6379): 1037-1042. doi: 10.1126/science.aar3246, the disclosure of each of which is incorporated by reference herein in its entirety.

An orthogonal cytokine system is one in which a cytokine and its receptor have been mutated such that they lose compatibility with their native (parental) partners yet retain the capacity to interact productively with one another. Such an orthogonal cytokine: receptor pair can thus be said to demonstrate “privileged” or “private” interactions. The approach of generating orthogonally constrained forms of cytokines and their receptors is of value for cell therapy because it provides a way to limit the scope of a cytokine's activity solely to the therapeutic (i.e., adoptively transferred) cells—these being the only cells expressing the engineered receptor and, consequently, the only cells capable of responding to the engineered cytokine.

Interleukin-21 (“IL-21”) is another pleiotropic cytokine with actions in a broad range of lymphoid, myeloid, and epithelial cells. IL-21 regulates both innate and adaptive immune responses; it not only has key roles in antitumor and antiviral responses, but also exerts major effects on inflammatory responses that promote the development of autoimmune diseases and inflammatory disorders. Spolski, R., Leonard, W. Interleukin-21: a double-edged sword with therapeutic potential. Nat Rev Drug Discov 13, 379-395 (2014). https://doi.org/10.1038/nrd4296. The three-dimensional structure of the natural human IL-21 cytokine: receptor complex is known. See Hamming O J, Kang L, Svensson A, et al. Crystal structure of interleukin-21 receptor (IL-21R) bound to IL-21 reveals that sugar chain interacting with WSXWS motif is integral part of IL-21R. J Biol Chem. 2012; 287 (12): 9454-9460. doi: 10.1074/jbc.M111.311084, the disclosure of which is incorporated by reference herein in its entirety.

IL-21 is of particular interest because it enhances cytotoxic T cell responses to viruses and tumors and can act in synergy with other cytokines, such as IL-2 or IL-15. IL-21 does this in part by promoting the persistence of T cells with a stem cell memory phenotype, which has been associated with beneficial outcomes in cell therapy settings. IL-21 is currently undergoing evaluation as a cancer therapeutic in multiple clinical trials. IL-21 also has significant potential utility in chimeric antigen receptor T (“CAR-T”) cell therapies, where it may help to overcome clinical failures due to poor expansion, anti-tumor efficacy, exhaustion, suppression, and persistence.

IL-21 is a gamma chain (γc) cytokine. A γc cytokine is any cytokine where the cognate cytokine receptor complex includes the common cytokine receptor gamma chain (“CD132” or “IL2RG”). γc cytokines signal through receptor complexes that contain the common gamma chain subunit. Besides IL-21, γc cytokines include IL-2, IL-4, IL-7, IL-9, and IL-15.

A need exists for a system in which both chains of the IL-21 receptor (IL-21Rα and IL2RG) are mutated such that their individual interactions with IL-21 are compromised. An orthogonal interleukin-21 cytokine (an “ortho-IL-21” or “ortho-IL-21 molecule,” or when referring to a specific ortho-IL-21 constructed as provided herein, a “CV,” as in “Cytokine Variant”) used in this instance would be similarly compromised in interactions with both wild-type IL-21Rα and wild-type IL2RG. This ortho-IL-21 would, however, signal normally when cells express an appropriate orthogonal interleukin-21 receptor alpha chain (an “ortho-IL-21Rα” or “ortho-IL-21Rα molecule,” or when referring to a specific ortho-IL-21Rα constructed as provided herein, an “RV,” as in “Receptor Variant”) and an appropriate orthogonal IL2RG (an “ortho-IL-21RG” or “ortho-IL-21RG molecule,” or when referring to a specific ortho-IL-21RG constructed as provided herein, an “R1-,” an “R2-,” or an “R3-”). One appeal of an orthogonal system of this sort is the opportunity it presents for modifying the cytoplasmic domains of both IL-21Rα and IL2RG and therefore providing increased versatility in the kind of signal ortho-IL-21 could generate in T cells. For example, because IL-10 signals through a receptor that does not involve an IL2RG (Ouyang, W. & O'Garra, A., Immunity 50, 871-891 (2019)), IL-10's signaling properties cannot be readily replicated in their entirety by a strategy involving modifications only to the cytoplasmic domain of IL-21Rα. However, the orthogonal system described herein would allow for the cytoplasmic domains of both IL-10RA and IL-10RB to be used in place of those normally present in IL-21Rα and IL2RG. Ortho-IL-21 would then have the potential to replicate IL-10 faithfully in the kind of signal IL-10 normally creates in T cells. IL-10 potentiates tumor-specific T cell responses in preclinical model systems, but its value as a therapeutic has proven limited, in part perhaps because of its immunosuppressive properties. Focusing the normal effect of IL-10 on therapeutic T cells using an ortho-IL-21 system (comprised of IL-21 receptors with intracellular domains from IL-21RA and IL-21RB) can provide a means for overcoming unwanted immunosuppression and increasing clinical efficacy. Similarly, an ortho-IL-21 system (comprised of IL-21 receptors with IL-21RA and IL-21RB intracellular domains) could also be used to provide primarily an immunosuppressive effect, if this was the desired goal.

In addition to an orthogonal cytokine-receptor system that employs variant forms of both the IL-21Rα and γc, an alternative orthogonal system may be generated that relies only on variation in γc.

The common gamma chain (γc) is critical for signal transduction in response to IL-2, IL-4, IL-7, IL-9, IL-15, and IL-21. Where it has been examined, there is similarity in the way the gamma chain binds to these cytokines, most notably for IL-21 and IL-2 (Abhiraman, G. C. et al. A structural blueprint for interleukin-21 signal modulation. Cell Rep. 2023; 42 (6): 112657). This similarity raises the possibility that a variant form of γc that is part of an orthogonal cytokine-receptor system based on IL-21 might also be appropriate for use in an alternative orthogonal cytokine-receptor system based on a different gamma cytokine, perhaps with minimal or no additional engineering of the gamma chain. This invention anticipates flexibility of this sort, and the therapeutic versatility it would allow.

### SUMMARY

In one aspect, an ortho-IL2RG (CD132 or common-γ or γc) chain is provided that is capable of rendering cells responsive to an ortho-cytokine. The ortho-cytokine may be ortho-IL-21 carrying substitutions in residues that impact IL2RG responses. In one aspect, IL2RG variation includes: Q127Y/H159P/P207G, Q127M/H159P/P207G, Q127Y/H159E/P207A, or Q127M/H159E/P207A. In one aspect, IL2RG variation may further include Y103L, Y103F, Y103W, or Y103I; N128E, N128T, N128G, or N128L; N206K, N206Y, N206Q, or N206T; L208S, L208A, L208F, or L208G; and G210I, G210A, G210Y, and combinations thereof. In one aspect, IL-21 variation includes: Q116Y, Q116F, Q116L, Q116M, Q116V, Q116K, or Q116R; H120Y or H120F; L123Y or L123F, and combinations thereof. In one aspect, the ortho-cytokine may be IL-2 carrying substitutions in residues that impact IL2RG responses. In one aspect, IL-2 variation includes: Q126Y, Q126F, Q126L, Q126M, Q126V, Q126K, or Q126R.

In one aspect, a method is provided for using the combination of ortho-IL2RG and ortho-IL-21 to influence the proliferation, differentiation, effector activity, and survival of cells, including lymphocytes, including engineered lymphocytes for use in cellular therapy, including CAR-T cells.

In one aspect, a method is provided for using the ortho-IL2RG in combination with ortho-IL-21Rα (CD360) engineered for impaired binding to natural (wild-type) IL-21 and enhanced binding to ortho-IL-21. In one aspect, the ortho-IL-21Rα includes a variant having an amino acid substitution numbered relative to SEQ ID NO: 347 of M70G. In one aspect, the ortho-IL-21 includes a variant having an amino acid substitution numbered relative to SEQ ID NO: 346 of H6L/R9K/M10L/K73I/P78L/G84E/P104V.

In one aspect, the ortho-IL-21Rα includes a variant with the native cytoplasmic domain replaced with one from: IL-2Rβ (CD122), IL-4Rα (CD124), IL-7Rα (CD127), IL-9Rα (CD129), IL10RA (CD210), or IL10RB (CDW210B). In one aspect, the ortho-IL2RG includes a variant with the native cytoplasmic domain replaced with one from: IL10RA (CD210) or IL10RB (CDW210B). In one aspect, the ortho-IL2RG includes a variant with the native cytoplasmic domain replaced with one from: IL-2Rβ (CD122), IL-4Rα (CD124), IL-7Rα (CD127), or IL-9Rα (CD129).

In one aspect, the expression of the ortho-IL2RG in cells may be by means of transposons, lentiviral (or other viral) vectors, or by nonviral means. In one aspect, cells expressing an ortho-IL2RG may be incubated with ortho-cytokines (IL-21 or IL-2) to impact their proliferation and phenotype. In one aspect, ortho-cytokines may be infused into patients carrying cells with ortho-IL2RG to impact their proliferation, differentiation, and effector functions, for example, to enhance anti-tumor functions or suppress deleterious immune responses.

In one aspect, engineered cells may be used as a source of ortho-cytokines during culture in vitro or after transfer to patients.

In one aspect, ortho-IL2RG may be expressed in cells from the native IL2RG locus by means of CRISPR/cas-dependent homology-directed replacement. In one aspect, ortho-cytokines may be used to cause the selective proliferation of cells expressing the ortho-IL2RG during in vitro culture. In one aspect, ortho-cytokines may be used to support—selectively—the survival, proliferation, and effector functions of cells expressing the ortho-IL2RG in patients. In one aspect, removal and/or depletion of the ortho-cytokines from patients may be used as a means of restraining their proliferation, differentiation, and effector functions, in the interest of patient safety, particularly when the engineered cells are associated with adverse effects.

### BRIEF DESCRIPTION OF THE FIGURES

The present invention may be more readily understood by reference to the following figures, wherein:

FIG. 1 shows a heat map representation of the responses of 96 Ba/F3 cell lines to the indicated six cytokines at two concentrations (200 and 35 ng/ml). The Ba/F3 cell lines all expressed the RV22 (M70G) variant of the IL-2Rα chain that allows strong responses to CV415, a variant of IL-21; they differed from one another in the IL2RG variant they co-expressed with this alpha chain (95 variants—Table 1—were used in the experiment; one cell line expressed the wild-type form of CD132/IL2RG). The cell lines were exposed to the cytokines overnight (in 384-well plates) prior to assaying luciferase activity present in the culture medium by luminometry (using the VLAR-1 reagent and Vargulin substrate from Targeting Systems, El Cajon, CA, USA). The cell lines all carried a transgene that expressed the luciferase from Cypridina noctiluca under the control of a STAT-3-responsive promoter.

FIG. 2 shows the responses made by Ba/F3 cells expressing IL2RG/CD132 variant R1-12 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 3 shows the responses made by Ba/F3 cells expressing IL2RG/CD132 variant R1-43 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 4 shows the responses made by Ba/F3 cells expressing IL2RG/CD132 variant R1-63 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 5 shows the responses made by Ba/F3 cells expressing IL2RG/CD132 variant R1-64 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 6 shows the responses made by Ba/F3 cells expressing IL2RG/CD132 variant R1-6 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 7 shows the responses made by Ba/F3 cells expressing the M70G variant of CD360/IL-21Rα (i.e., RV22) to the indicated cytokines. The cells used in this experiment were not transfected with an IL2RG transgene and thus only expressed the endogenous (mouse) form of CD132. As in FIG. 1, the cells were exposed to the cytokines overnight prior to luminometry. Quadruplicate determinations were made at each of the cytokine concentrations.

FIG. 8 shows the responses made by Ba/F3 cells expressing the wild-type form of CD360/IL-21Rα to the indicated cytokines. The cells used in this experiment were not transfected with an IL2RG transgene and thus only expressed the endogenous (mouse) form of CD132. As in FIG. 1, the cells were exposed to the cytokines overnight prior to luminometry. Quadruplicate determinations were made at each of the cytokine concentrations.

FIG. 9 shows the responses made by Ba/F3 cells expressing wild-type human IL2RG/CD132 to the indicated cytokines. The cells used in this experiment were derived from the same cultures as those used for the experiment that yielded the results presented in FIG. 1; they expressed the M70G variant of CD360/IL-21Rα (i.e., RV22). As in FIG. 1, the cells were exposed to the cytokines overnight prior to luminometry. Quadruplicate determinations were made at each of the cytokine concentrations.

FIG. 10 shows box violin plots, each one representing the responses made by 96 Ba/F3 cell lines to a single concentration (17 ng/ml) of the indicated cytokines. The cell line collection used in this experiment was a replica of the collection used for FIG. 1. As in FIG. 1, the cells were exposed to the cytokines overnight prior to luminometry. Singlet determinations were made in each case.

FIG. 11 shows box violin plots, each one representing the responses made by 96 Ba/F3 cell lines to a single concentration (17 ng/ml) of the indicated cytokines. The cell line collection used in this experiment was a replica of the collection used for FIG. 1. As in FIG. 1, the cells were exposed to the cytokines overnight prior to luminometry. Singlet determinations were made in each case.

FIG. 12 (left-hand panel) shows detail at the top ends of two of the box violin plots from FIG. 10. Each of the individual data points in the expanded parts of the two distributions is labeled with the substitution present in the involved IL2RG variant. The right hand panel shows a bivariate plot of a subset of the datapoints from a Z-score transformation of the indicated distributions (IL-21-WT and CV415-Q116Y) from FIG. 10.

FIG. 13 shows a bivariate plot of a subset of the datapoints from a Z-score transformation of the indicated distributions (IL-21-WT and CV415-Q116F) from FIG. 10.

FIG. 14 shows box violin plots, each one representing the responses made by 96 Ba/F3 cell lines to a single concentration (17 ng/ml) of the indicated cytokines. The cell line collection used in this experiment involved the IL2RG variants listed in Table 4. As in FIG. 1, the cells were exposed to the cytokines overnight prior to luminometry. Singlet determinations were made in each case.

FIG. 15 shows box violin plots, each one representing the responses made by an individual Ba/F3 cell line to a single concentration (17 ng/ml) of the twenty-one cytokines represented in FIG. 14. The cell line collection used in this experiment involved the IL2RG variants listed in Table 4. As in FIG. 1, the cells were exposed to the cytokines overnight prior to luminometry. Singlet determinations were made in each case.

FIG. 16 shows the responses made by 96 Ba/F3 cell lines (involving the IL2RG variants listed in Table 4) to a titration of IL-21-WT. As in FIG. 1, the cells were exposed to the cytokines overnight prior to luminometry. Duplicate determinations were made in each case. The responses made by twelve cell lines are highlighted.

FIG. 17 shows the responses made by 96 Ba/F3 cell lines (involving the IL2RG variants listed in Table 4) to a titration of CV415. As in FIG. 1, the cells were exposed to the cytokines overnight prior to luminometry. Duplicate determinations were made in each case. The responses made by twelve cell lines are highlighted.

FIG. 18 shows the responses made by 96 Ba/F3 cell lines (involving the IL2RG variants listed in Table 4) to a titration of CV415-Q116Y. As in FIG. 1, the cells were exposed to the cytokines overnight prior to luminometry. Duplicate determinations were made in each case. The responses made by twelve cell lines are highlighted.

FIG. 19 shows the responses made by 96 Ba/F3 cell lines (involving the IL2RG variants listed in Table 4) to a titration of CV415-Q116L. As in FIG. 1, the cells were exposed to the cytokines overnight prior to luminometry. Duplicate determinations were made in each case. The responses made by twelve cell lines are highlighted.

FIG. 20 shows the responses made by 48 Ba/F3 cell lines (involving IL2RG variants from among those listed in Table 5) to a single concentration (0.1 ng/ml) of the twelve cytokines listed in Table 6. As in FIG. 1, the cells were exposed to the cytokines overnight prior to luminometry. Singlet determinations were made in each case. Half of the cell lines expressed IL2RG variants based on R2-168, and the other half based on R2-182, as indicated. The substitutions present in the IL-2RG variants are indicated on the X-axis.

FIG. 21 shows the responses made by 48 Ba/F3 cell lines (involving IL2RG variants from among those listed in Table 5) to a single concentration (0.1 ng/ml) of the twelve cytokines listed in Table 6. As in FIG. 1, the cells were exposed to the cytokines overnight prior to luminometry. Singlet determinations were made in each case. Half of the cell lines expressed IL2RG variants based on R2-168, and the other half based on R2-173, as indicated. The substitutions present in the IL-2RG variants are indicated on the X-axis.

FIG. 22 shows the responses made by 48 Ba/F3 cell lines (involving IL2RG variants from among those listed in Table 5) to a single concentration (0.1 ng/ml) of the twelve cytokines listed in Table 6. As in FIG. 1, the cells were exposed to the cytokines overnight prior to luminometry. Singlet determinations were made in each case. Half of the cell lines expressed IL2RG variants based on R2-168, and the other half based on R2-177, as indicated. The substitutions present in the IL-2RG variants are indicated on the X-axis.

FIG. 23 shows bivariate plots of data from the experiment depicted in FIG. 22. Each plot shows the responses made by forty-eight Ba/F3 cell lines to a single concentration (0.1 ng/ml) of the indicated cytokines (CV415-Q116Y, IL-21-WT, CV415, and CV415-Q116F, clockwise for the four graphs from the one at top left, respectively). The axis in each graph corresponds to the origin of the IL-2RG variants plotted (i.e., whether based on R2-168 or R2-177), and the individual data points are labeled with the substitutions present in the involved variants.

FIG. 24 shows bivariate plots of data from the experiment depicted in FIG. 22. Each plot shows the responses made by forty-eight Ba/F3 cell lines to a single concentration (0.1 ng/ml) of the indicated cytokines (CV415-Q116F_H120Y_L123F at left and CV415-Q116F_H120F_L123F, at right). The axis in each graph corresponds to the origin of the IL-2RG variants plotted (i.e., whether based on R2-168 or R2-177), and the individual data points are labeled with the substitutions present in the involved variants.

FIG. 25 shows the responses made by 24 Ba/F3 cell lines (involving IL2RG variants from among those listed in Table 5 and three control cell lines at the extreme right) to a single concentration (8 ng/ml) of the twelve cytokines listed in Table 6. As in FIG. 1, the cells were exposed to the cytokines overnight prior to luminometry. The origin (i.e., whether from R2-168, R2-173, R2-177, or R2-182) and the substitutions present in the involved IL2RG variants are provided on the X-axis. Each box violin plot represents the responses made by a single cell line to twelve cytokines.

FIG. 26 shows the responses made by 45 Ba/F3 cell lines (involving IL2RG variants from among those listed in Table 5) to a single concentration (4 ng/ml) of the twelve cytokines listed in Table 6. As in FIG. 1, the cells were exposed to the cytokines overnight prior to luminometry. Singlet determinations were made in each case. Twenty-three of the cell lines expressed RV22 and twenty-two expressed wild-type IL-21Rα, as indicated. In one case (RV22Δcyt_WTγ), the cell line expressed a form of RV22 lacking its cytoplasmic tail. Three cell lines (at the extreme right) expressed the wild-type form of IL2RG (WTγ). Each box violin plot represents the responses made by a single cell line to twelve cytokines.

FIG. 27 shows the responses made by 45 Ba/F3 cell lines (involving IL2RG variants from among those listed in Table 5) to a single concentration (4 ng/ml) of either IL-21-WT (top) or CV415 (bottom). As in FIG. 1, the cells were exposed to the cytokines overnight prior to luminometry. Singlet determinations were made in each case. Twenty-three of the cell lines expressed RV22, and 22 expressed wild-type IL-21Rα, as indicated. In one case (RV22Δcyt_WTγ), the cell line expressed a form of RV22 lacking its cytoplasmic tail. Three cell lines (at the extreme right) expressed the wild-type form of IL2RG (WTγ).

FIG. 28 shows the responses made by the 45 Ba/F3 cell lines represented in FIGS. 26 and 27 to titrations of the indicated cytokines. Twenty-three of the cell lines expressed RV22 (bottom three graphs), and 22 expressed wild-type IL-21Rα (top three graphs). As in FIG. 1, the cells were exposed to the cytokines overnight prior to luminometry. Singlet determinations were made in each case. The responses made by seven cell lines are highlighted.

FIG. 29 shows the responses made by six Ba/F3 cell lines to the indicated cytokines (IL-21-CV415-YYY is a cytokine variant CV415 carrying Q116Y, H120Y, and L123Y substitutions). The cell lines expressed wild-type IL-21Rα or RV22 (M70G) as indicated. One cell line expressed a form of RV22 lacking its cytoplasmic tail (RV22Δcyt). The cells expressed either the wild-type form of IL2RG or variant R3-202 (which is R2-168 carrying the Y103F substitution).

FIG. 30 shows the responses of six cell lines to the wild-type form of IL-21 or a variant of this cytokine carrying the Q116Y substitution. The cell lines expressed wild-type IL-21Rα or RV22 as indicated. One of the cell lines expressed a variant of RV22 lacking its cytoplasmic tail (RV22Δcyt). Three of the cell lines were positive for ectopic IL2RG expression (the wild-type form, variant R2-172 or variant R2-177, as indicated); the other three cell lines only expressed endogenous mouse CD132. As in FIG. 1, the cells were exposed to the cytokines overnight prior to luminometry. Duplicate determinations were made in each case.

FIG. 31 shows the responses of six cell lines to either a variant form of IL-21 carrying the Q116F substitution or a variant form of IL-21 carrying the Q116Y substitution. The cell lines expressed wild-type IL-21Rα or RV22 as indicated. One of the cell lines expressed a variant of RV22 lacking its cytoplasmic tail (RV22Δcyt). Three of the cell lines were positive for ectopic IL2RG expression (the wild-type form, variant R2-172 or variant R2-177, as indicated); the other three cell lines only expressed endogenous mouse CD132. As in FIG. 1, the cells were exposed to the cytokines overnight prior to luminometry. Duplicate determinations were made in each case.

### DETAILED DESCRIPTION

In one aspect, an ortho-IL2RG is provided that has impaired binding to native IL-21 (SEQ ID NO: 346) but binds to an ortho-IL-21, the ortho-IL2RG having a modified amino acid sequence derived from wild-type human IL2RG (SEQ ID NO: 348) comprising a mutation of one or more amino acids that contact IL-21. In some aspects, the ortho-IL2RG comprises a mutation at one or more of the amino acids at positions N44, V45, Y103, Q104, Q127, N128, H159, L161, E162, N206, P207, L208, G210, and S211 (numbered relative to SEQ ID NO: 348).

In one aspect, an ortho-IL-21 is provided that is incapable of inducing a signaling response in cells unless they express an appropriate ortho-IL-21Rα and ortho-IL2RG. To accomplish this, a candidate ortho-IL-21—with absent or reduced binding to native IL-21Rα (SEQ ID NO: 347) and specificity for a candidate ortho-IL-21Rα—has been mutated to effect compromised binding to native IL2RG. Substitutions have been identified in IL-21 that diminish or completely abrogate binding to IL2RG with minimal impact on IL-21Rα binding. For example, Q116D and Q116L are substitutions that block IL-21 function because of this kind of effect. Indeed, IL-21 carrying Q116D and H120D functions as an IL-21 antagonist, as does a form of the cytokine carrying the combination of Q116D and L123D. This antagonism derives from the capacity of the cytokine to engage with IL-21Rα yet fail to initiate signaling because of impaired affinity for IL2RG.

Ortho-IL-21 carrying substitutions at positions Glutamine-116 and/or Histidine-120 and/or Leucine-123 can be generated and used in screening assays to identify candidate ortho-IL2RG molecules. The screening procedure can involve HeLa cells because HeLa cells do not express endogenous IL2RG but can make a signaling response when transfected to express it. Alternatively, Ba/F3 cells can be used with appropriate provisions made for the fact that these cells express the mouse orthologue of IL2RG, which can form functional receptors for human IL-21 when paired with human IL-21Rα.

A collection of IL2RG variants (e.g., ˜100) can be generated, each differing from one another by a single amino acid substitution. The substitutions are made based on two kinds of considerations. The first consideration is comparisons between IL2RG orthologs from a range of species and the identification of recurring conservative substitutions. The second consideration is substitution at residues implicated in the IL2RG: IL-21 interaction by alanine-scanning mutagenesis or by structural models informed by published structures of IL2RG-containing receptor-cytokine complexes. Such residues include Asparagine-44, Tyrosine-103, Glutamine-127, Asparagine-128, Histidine-159, Leucine-161, Glutamic acid-162, Asparagine-206, Proline-207, Leucine-208, Glycine-210, and Serine-211.

To screen for ortho-IL2RG interactions, the native form of the cytokine can be used as the starting point for mutagenesis. If instead a candidate ortho-IL-21 is used, the properties of IL2RG substitutions can be assessed directly in the context of an ortho-IL-21 system. In either scenario, the cytokine would be diversified by substitutions at any, or all, of Glutamine-116, Histidine-120, and Leucine-123 to compromise binding to native IL2RG. Alternative substitutions could also be made based on the structure of IL-21 bound to IL-2Rα and γc (Abhiraman, G. C. et al. A structural blueprint for interleukin-21 signal modulation. Cell Rep. 2023; 42 (6): 112657).

Ortho-IL-21 can be incubated with the small library of HeLa or Ba/F3 cell lines, each of which expresses an invariant candidate ortho-IL-21Rα in combination with distinct candidate ortho-IL2RG molecules (e.g., a collection of 96 such cell lines, differing in the particular ortho-IL2RG they each express). One experimental system involves testing multiple concentrations of each ortho-IL-21 candidate with each cell line such that dose-response relationships can be established and compared.

A screening strategy of the sort just outlined can identify substitutions in IL2RG that are functionally complementary to specific substitutions in IL-21. Crucially, the strategy is focused on substitutions in IL-21 that impair binding between the cytokine and native—i.e., nonvariant—IL2RG so that complementation restores function to a cytokine that is otherwise partially or fully disabled.

The screening strategy can include subsequent phases in which positively scoring substitutions are combined to engineer a variant form of IL2RG that has improved complementation activity. The inclusion of multiple ortho-IL-21 candidates (differing in their substitutions for Glutamine-116, Histidine-120, and Leucine-123) at the outset is expected to increase the likelihood that the IL2RG mutagenesis strategy will succeed. It can also permit the isolation of mutually orthogonal systems.

An efficient, fully orthogonal cytokine system can be comprised of a version of IL-21 that retains weak binding to the native forms of IL-21Rα and/or IL2RG, but where there is an additive or synergistic effect when the two interactions are both compromised. This possibility is significant in the sense that it can prove simpler to isolate an orthogonal system of this sort than one in which both kinds of interaction (IL-21: IL-21Rα and IL-21: IL2RG) are strictly orthogonal.

### Definitions

U.S. Nonprovisional patent application Ser. No. 18/304,172, including the “Definitions” set forth in paragraphs [0028]-[0045], is incorporated by reference herein in its entirety.

### EXAMPLES

Residues in human IL2RG that are involved in contacting the cytokines IL-2 and IL-4 include Y103, Q127, N128, H159, N206, N207, L208, G210, and S211 (See Wang, X, Rickert, M, and Garcia, K C. Science 310, 1159-63; doi: 10.1126/science.1117893 and LaPorte, S L, Juo, Z S, Vaclavikova, J, Colf, L A, Qi, X, Heller, N M, Keegan, A D, and Garcia, K C. Cell 132, 259-72; doi: 10.1016/j.cell.2007.12.030). These residues were varied in isolation (i.e., single substitutions in otherwise native protein sequence contexts) in each of the 95 ortho-IL2RG candidates shown in Table 1 (each numbered relative to SEQ ID NO: 348).

TABLE 1

| | ortho-IL2RG |
|---|---|
| SEQ ID NO: | candidate_Substitution |
| 1 | R1-1_Y103F |
| 2 | R1-2_Y103W |
| 3 | R1-3_Y103M |
| 4 | R1-4_Y103L |
| 5 | R1-5_Y103I |
| 6 | R1-6_Y103Q |
| 7 | R1-7_Y103A |
| 8 | R1-8_Y103P |
| 9 | R1-9_Y103G |
| 10 | R1-10_Y103H |
| 11 | R1-11_Q127N |
| 12 | R1-12_Q127Y |
| 13 | R1-13_Q127K |
| 14 | R1-14_Q127E |
| 15 | R1-15_Q127R |
| 16 | R1-16_Q127D |
| 17 | R1-17_Q127S |
| 18 | R1-18_Q127M |
| 19 | R1-19_Q127H |
| 20 | R1-20_Q127G |
| 21 | R1-21_Q127P |
| 22 | R1-22_N128L |
| 23 | R1-23_N128E |
| 24 | R1-24_N128R |
| 25 | R1-25_N128S |
| 26 | R1-26_N128T |
| 27 | R1-27_N128K |
| 28 | R1-28_N128H |
| 29 | R1-29_N128G |
| 30 | R1-30_N128A |
| 31 | R1-31_N128D |
| 32 | R1-32_N128Q |
| 33 | R1-33_N128P |
| 34 | R1-34_H159Q |
| 35 | R1-35_H159K |
| 36 | R1-36_H159R |
| 37 | R1-37_H159V |
| 38 | R1-38_H159I |
| 39 | R1-39_H159N |
| 40 | R1-40_H159E |
| 41 | R1-41_H159Y |
| 42 | R1-42_H159A |
| 43 | R1-43_H159P |
| 44 | R1-44_N206S |
| 45 | R1-45_N206A |
| 46 | R1-46_N206H |
| 47 | R1-47_N206T |
| 48 | R1-48_N206G |
| 49 | R1-49_N206E |
| 50 | R1-50_N206D |
| 51 | R1-51_N206I |
| 52 | R1-52_N206Q |
| 53 | R1-53_N206Y |
| 54 | R1-54_N206K |
| 55 | R1-55_N206P |
| 56 | R1-56_P207N |
| 57 | R1-57_P207S |
| 58 | R1-58_P207E |
| 59 | R1-59_P207K |
| 60 | R1-60_P207T |
| 61 | R1-61_P207Q |
| 62 | R1-62_P207I |
| 63 | R1-63_P207A |
| 64 | R1-64_P207G |
| 65 | R1-65_P207V |
| 66 | R1-66_L208Y |
| 67 | R1-67_L208F |
| 68 | R1-68_L208S |
| 69 | R1-69_L208V |
| 70 | R1-70_L208N |
| 71 | R1-71_L208R |
| 72 | R1-72_L208I |
| 73 | R1-73_L208A |
| 74 | R1-74_L208G |
| 75 | R1-75_L208P |
| 76 | R1-76_G210A |
| 77 | R1-77_G210I |
| 78 | R1-78_G210L |
| 79 | R1-79_G210M |
| 80 | R1-80_G210T |
| 81 | R1-81_G210S |
| 82 | R1-82_G210Y |
| 83 | R1-83_G210W |
| 84 | R1-84_G210F |
| 85 | R1-85_G210V |
| 86 | R1-86_S211N |
| 87 | R1-87_S211T |
| 88 | R1-88_S211R |
| 89 | R1-89_S211M |
| 90 | R1-90_S211I |
| 91 | R1-91_S211G |
| 92 | R1-92_S211Y |
| 93 | R1-93_S211W |
| 94 | R1-94_S211F |
| 95 | R1-95_S211V |

Transgenes to express the variants were embedded in LeapIn™ transposons together with a drug resistance gene (conferring resistance to hygromycin B). The transposons were in turn contained within plasmids to permit their replication in E. coli.

Plasmids carrying the ortho-IL2RG-encoding transposons were transfected into Ba/F3 cells together with mRNA encoding LeapIn Transposase® enzyme. About 1 μg of DNA and 0.2 μg of mRNA were used in each transfection. A Lonza 4D Nucleofector instrument was used with the X module and the SG cell line kit according to the manufacturer's instructions.

The Ba/F3 cells used in the transfections had been previously rendered positive for expression of the ortho-IL-21Rα “RV22” (SEQ ID NO: 349), which comprises an amino acid substitution, numbered relative to SEQ ID NO: 347 (the human IL-21Rα ectodomain in mature form lacking the signal peptide), of M70G. This was accomplished by transfecting the cells with a plasmid carrying a transposon containing four transgenes: one expressing RV22; a second expressing the puromycin N-acetyl transferase; a third conferring STAT3-responsive expression of the Cypridina noctiluca luciferase; and a fourth conferring constitutive expression of a luciferase from the Brazilian click beetle Pyrearinus termitilluminans.

The Ba/F3 cells were subjected to puromycin selection (2 μg/ml) after the first transfection with the RV22-encoding transposon. They were subsequently subjected to combined puromycin (2 μg/ml) and hygromycin B (2.5 μg/ml) selection after transfection with the ortho-IL2RG-encoding transposons. A transposon encoding wild-type human IL2RG (SEQ ID NO: 348) was included as a control. Expression of RV22 and the ortho-IL2RG candidates was quantified by flow cytometry with only three ortho-IL2RG candidates showing significantly impaired expression: (1) “R1-33,” which comprises an amino acid substitution, numbered relative to SEQ ID NO: 348, of N128P, and is represented by SEQ ID NO: 33; (2) “R1-49,” which comprises an amino acid substitution, numbered relative to SEQ ID NO: 348, of N206E, and is represented by SEQ ID NO: 49; and (3) “R1-55,” which comprises an amino acid substitution, numbered relative to SEQ ID NO: 348, of N206P, and is represented by SEQ ID NO: 55.

Twelve ortho-IL-21 candidates were generated for screening with the IL2RG candidate cell line collection. Two controls were also tested: wild-type IL-21 (SEQ ID NO: 350) and “CV415” (SEQ ID NO: 96), which comprises amino acid substitutions, numbered relative to SEQ ID NO: 346, of H6L/R9K/M10L/K73I/P78L/G84E/P104V. The phrase “numbered relative to SEQ ID NO: 346” means, for numbering purposes, to disregard any epitope tags and signal peptides (the sequence of the signal peptide and epitope tags used for IL-21 expression are provided in isolation in SEQ ID NO: 351). The twelve ortho-IL-21 candidates were variants of CV415 carrying substitutions at residue Q116 in isolation or combined with additional substitutions at either H120 or L123, as shown in Table 2:

TABLE 2

| ortho-IL-21 candidate | SEQ ID NO: |
|---|---|
| CV415 | 96 |
| CV415-Q116D | 97 |
| CV415-Q116D/L123D | 98 |
| CV415-Q116D/H120D | 99 |
| CV415-Q116Y | 100 |
| CV415-Q116Y/L123D | 101 |
| CV415-Q116Y/H120D | 102 |
| CV415-Q116K | 103 |
| CV415-Q116K/L123D | 104 |
| CV415-Q116K/H120D | 105 |
| CV415-Q116L | 106 |
| CV415-Q116L/L123D | 107 |
| CV415-Q116L/H120D | 352 |

The collection of 96 Ba/F3 cell lines was expanded to confluency in five 96-well flat-bottomed plates. The cultures were subsequently cultured overnight (˜20 hours) in the absence of serum before plating in the presence of four concentrations (300, 50, 8.3, and 1.4 ng/ml) of each of the ortho-IL-21 candidates. Singlet determinations were used in each case such that the entire collection of 96 cell lines was tested against the four concentrations of each ortho-IL-21 candidate using a single 384-well plate. Each assay involved approximately 20,000 cells in a final volume of 60 μl per well. Fourteen plates were used to assay the twelve ortho-IL-21 candidates and the two controls.

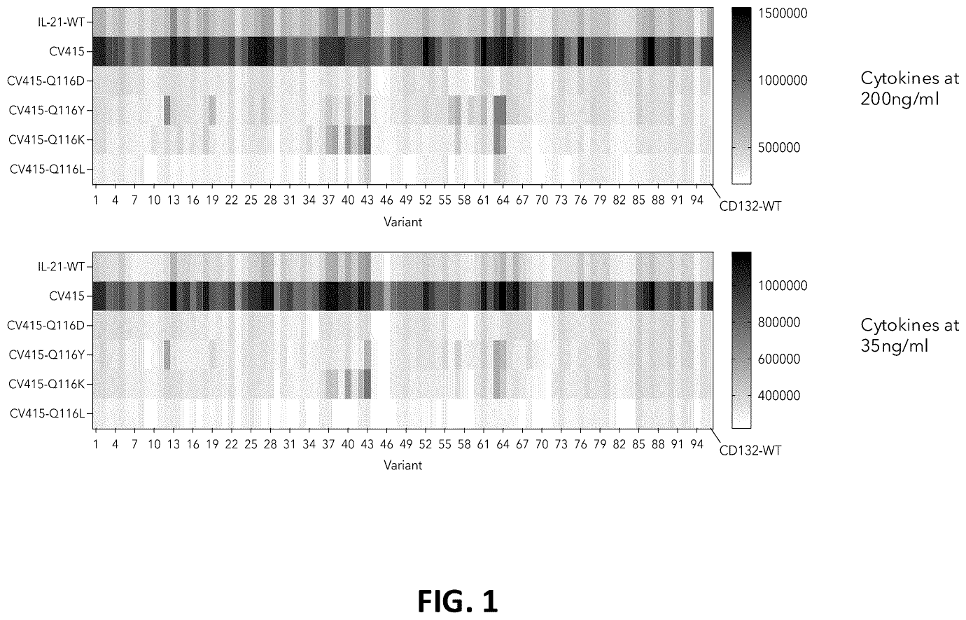

Ortho-IL-21 candidates carrying Q116D, H120D, or L123D substitutions were almost entirely inactive against the panel of cell lines. Three cell lines showed minimal responses to the Q116L form of CV415 at the highest concentration tested. By contrast, multiple cell lines responded to the Q116Y and Q116K forms of CV415 (FIG. 1). These cell lines included those that expressed ortho-IL2RG candidates that carried amino acid substitutions including Q127Y, Q127E, Q127M, Q127H, H159Q, H159V, H159I, H159E, H159Y, H159A, H159P, P207N, P207S, P207Q, P207A, and P207G.

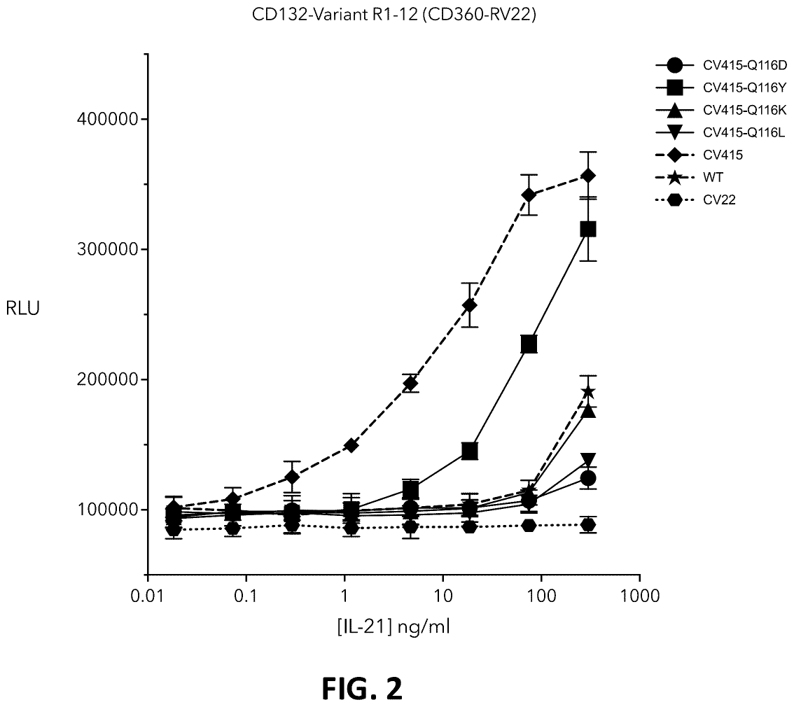

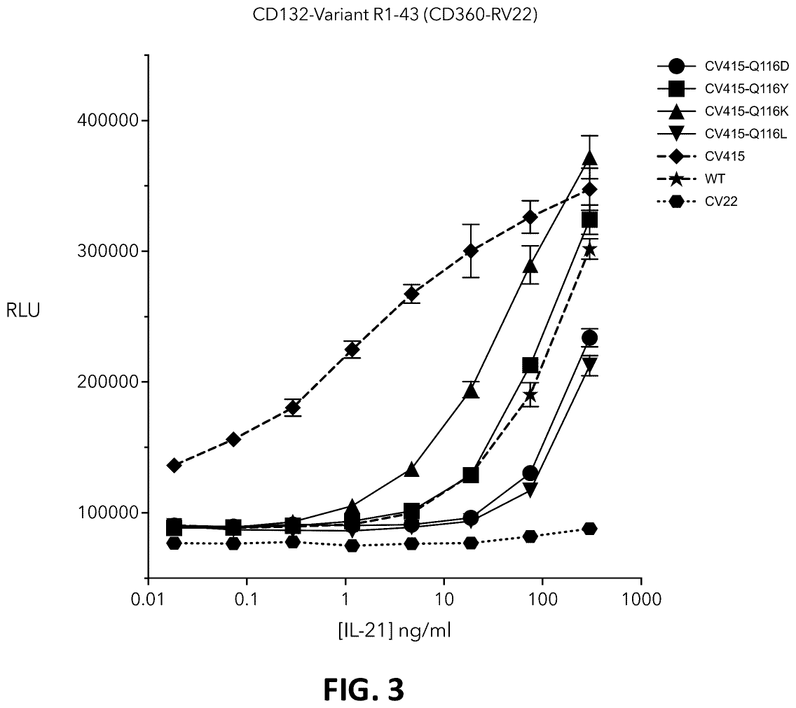

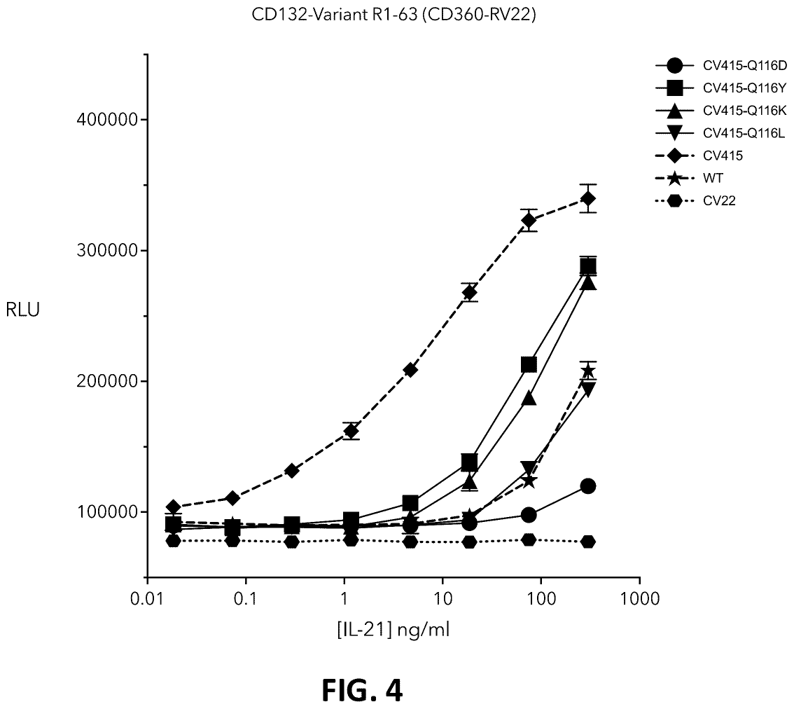

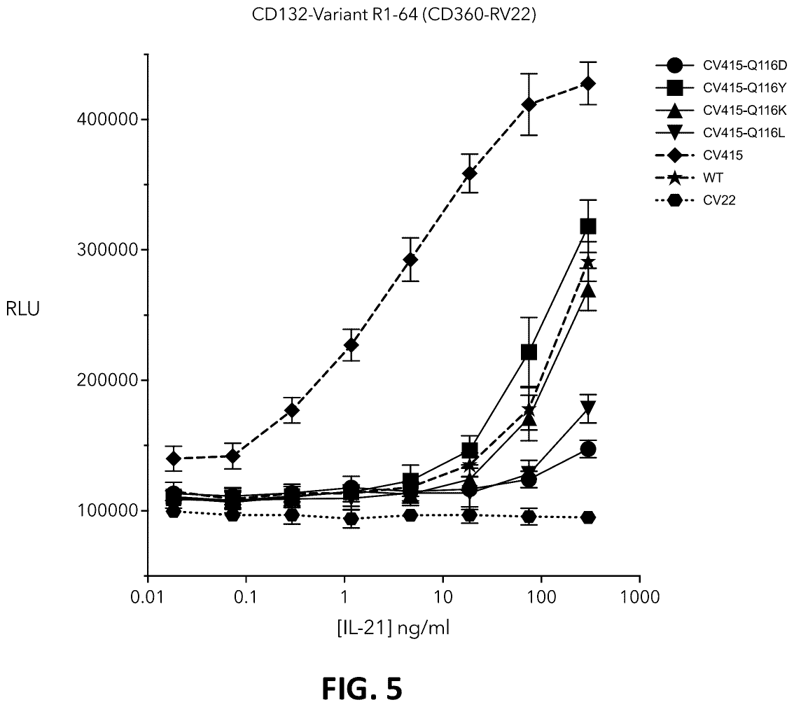

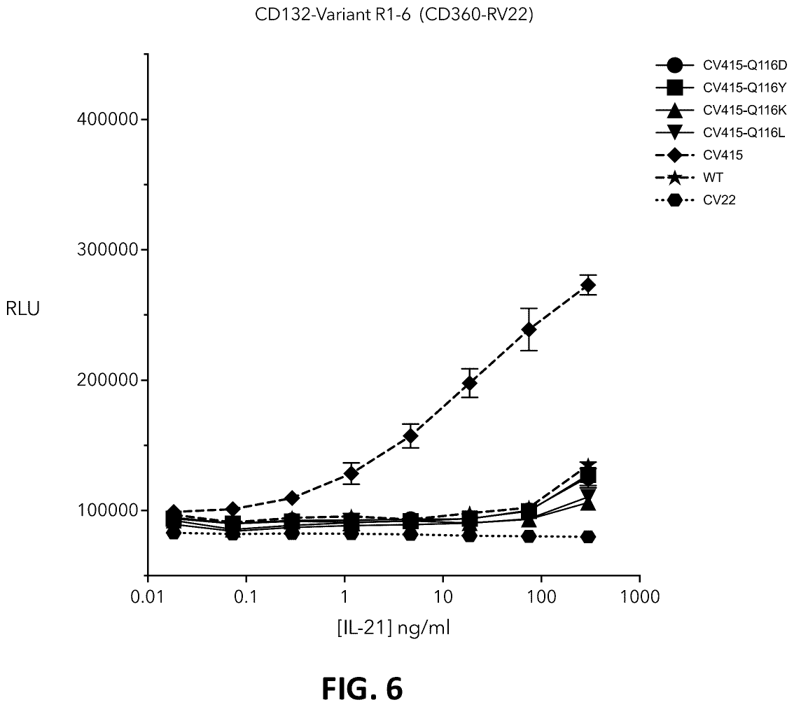

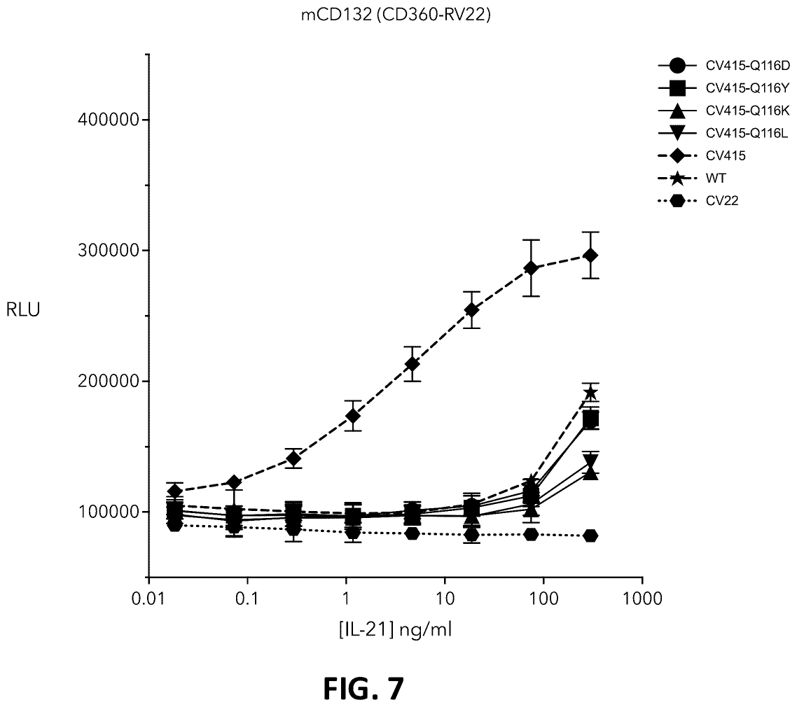

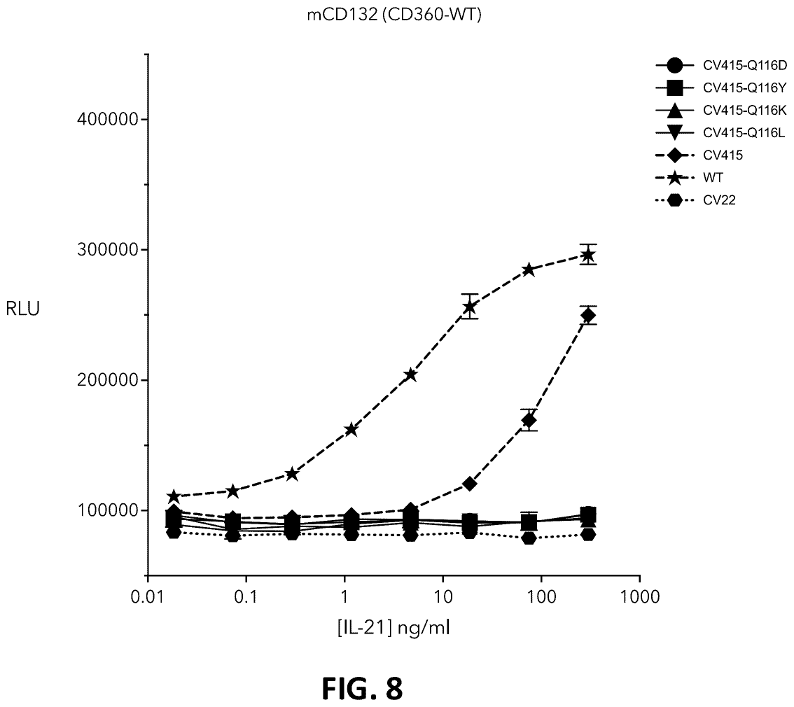

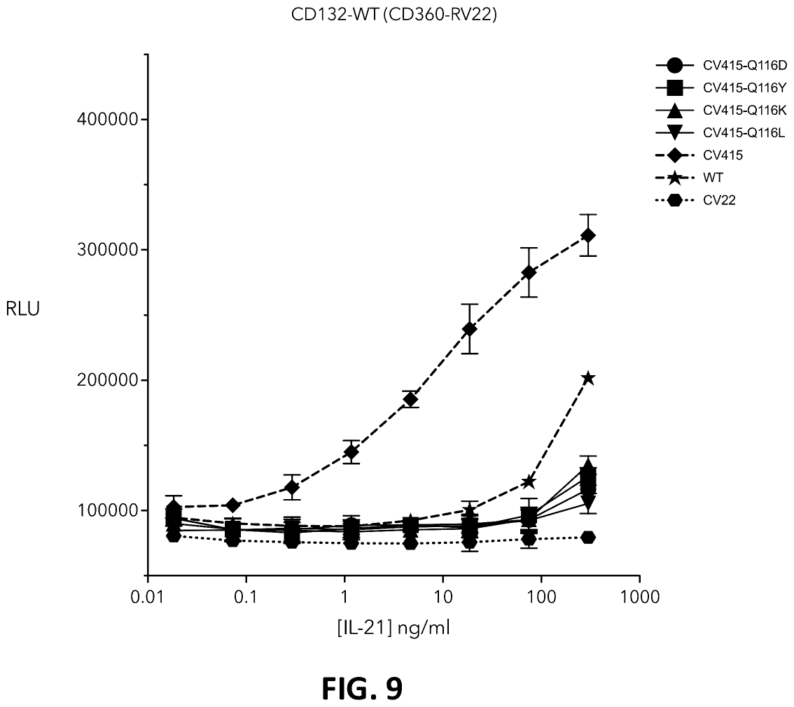

Ortho-IL-21 candidates carrying the Q116D, Q116Y, Q116K, or Q116L substitutions were selected for retesting with a subset of the responding cell lines. This experiment involved more extensive titrations of the cytokines and quadruplicate determinations (FIGS. 2-9). Three control cytokines were used: wild-type IL-21, CV415, and a functionally inactive form of IL-21 carrying R5Q and R76A substitutions (SEQ ID NO: 353). Three control cell lines were also included: one expressing RV22 in combination with wild-type human CD132 (FIG. 9), one expressing RV22 in combination with endogenous mouse CD132 (FIG. 7), and one expressing the wild-type form of IL-21Rα again in combination with endogenous mouse CD132 (FIG. 8). Key findings included the observation that RV22-positive cells expressing only the endogenous form of mouse IL2RG or those that expressed wild-type human IL2RG from a transposon made minimal or undetectable responses to the retested ortho-IL-21 candidates (FIGS. 7 and 9). Similarly, cells expressing wild-type IL-21Rα failed to respond to the ortho-IL-21 candidates (FIG. 8). By contrast, there were clear responses made by multiple cell lines expressing ortho-IL2RG candidates, most notably, but not solely, those carrying the H159P (R1-43; FIG. 3) or P207A (R1-63; FIG. 4) substitutions.

Although some of the ortho-IL2RG candidates were associated with responsiveness to ortho-IL-21 candidates carrying substitutions at residue 116, the responsiveness they conferred was reduced (in terms of EC50) relative to that of control cytokines (such as wild-type IL-21 engaging with cells expressing wild-type IL-21Rα or CV415 engaging with cells expressing RV22). To try to address this and create a form of ortho-IL2RG associated with full or nearly full responsiveness, two steps were taken. One was to test a broader range of substitutions at residues 116, 120, and 123 (Table 3).

TABLE 3

| SEQ ID NO: | ortho-IL-21 candidate |
|---|---|
| 108 | CV415_Q116A |
| 109 | CV415_Q116R |
| 110 | CV415_Q116N |
| 111 | CV415_Q116E |
| 112 | CV415_Q116G |
| 113 | CV415_Q116H |
| 114 | CV415_Q116I |
| 115 | CV415_Q116M |
| 116 | CV415_Q116F |
| 117 | CV415_Q116P |
| 118 | CV415_Q116S |
| 119 | CV415_Q116T |
| 120 | CV415_Q116W |
| 121 | CV415_Q116V |
| 122 | CV415_H120Q |
| 123 | CV415_H120R |
| 124 | CV415_H120V |
| 125 | CV415_H120I |
| 126 | CV415_H120N |
| 127 | CV415_H120F |
| 128 | CV415_H120Y |
| 129 | CV415_H120A |
| 130 | CV415_H120S |
| 131 | CV415_H120K |
| 132 | CV415_L123Y |
| 133 | CV415_L123F |
| 134 | CV415_L123S |
| 135 | CV415_L123V |
| 136 | CV415_L123N |
| 137 | CV415_L123R |
| 138 | CV415_L123I |
| 139 | CV415_L123A |
| 140 | CV415_L123G |
| 141 | CV415_L123M |
| 142 | IL-21-WT_Q116K |
| 143 | IL-21-WT_Q116Y |
| 144 | IL-21-WT_Q116F |

The other was to test a panel of ortho-IL2RG candidates carrying double or triple substitutions comprised of combinations of substitutions that were associated with increased responsiveness in the experiments just described (Table 4).

TABLE 4

| SEQ ID NO: | ortho-IL2RG candidate |
|---|---|
| 145 | R2-101_Q127Y_H159Q |
| 146 | R2-102_Q127Y_H159V |
| 147 | R2-103_Q127Y_H159I |
| 148 | R2-104_Q127Y_H159E |
| 149 | R2-105_Q127Y_H159Y |
| 150 | R2-106_Q127Y_H159A |
| 151 | R2-107_Q127Y_H159P |
| 152 | R2-108_Q127E_H159Q |
| 153 | R2-109_Q127E_H159V |
| 154 | R2-110_Q127E_H159I |
| 155 | R2-111_Q127E_H159E |
| 156 | R2-112_Q127E_H159Y |
| 157 | R2-113_Q127E_H159A |
| 158 | R2-114_Q127E_H159P |
| 159 | R2-115_Q127M_H159Q |
| 160 | R2-116_Q127M_H159V |
| 161 | R2-117_Q127M_H159I |
| 162 | R2-118_Q127M_H159E |
| 163 | R2-119_Q127M_H159Y |
| 164 | R2-120_Q127M_H159A |
| 165 | R2-121_Q127M_H159P |
| 166 | R2-122_Q127H_H159Q |
| 167 | R2-123_Q127H_H159V |
| 168 | R2-124_Q127H_H159I |
| 169 | R2-125_Q127H_H159E |
| 170 | R2-126_Q127H_H159Y |
| 171 | R2-127_Q127H_H159A |
| 172 | R2-128_Q127H_H159P |
| 173 | R2-129_H159Q_P207N |
| 174 | R2-130_H159Q_P207S |
| 175 | R2-131_H159Q_P207Q |
| 176 | R2-132_H159Q_P207A |
| 177 | R2-133_H159Q_P207G |
| 178 | R2-134_H159V_P207N |
| 179 | R2-135_H159V_P207S |
| 180 | R2-136_H159V_P207Q |
| 181 | R2-137_H159V_P207A |
| 182 | R2-138_H159V_P207G |
| 183 | R2-139_H159I_P207N |
| 184 | R2-140_H159I_P207S |
| 185 | R2-141_H159I_P207Q |
| 186 | R2-142_H159I_P207A |
| 187 | R2-143_H159I_P207G |
| 188 | R2-144_H159E_P207N |
| 189 | R2-145_H159E_P207S |
| 190 | R2-146_H159E_P207Q |
| 191 | R2-147_H159E_P207A |
| 192 | R2-148_H159E_P207G |
| 193 | R2-149_H159Y_P207N |
| 194 | R2-150_H159Y_P207S |
| 195 | R2-151_H159Y_P207Q |
| 196 | R2-152_H159Y_P207A |
| 197 | R2-153_H159Y_P207G |
| 198 | R2-154_H159A_P207N |
| 199 | R2-155_H159A_P207S |
| 200 | R2-156_H159A_P207Q |
| 201 | R2-157_H159A_P207A |
| 202 | R2-158_H159A_P207G |
| 203 | R2-159_H159P_P207N |
| 204 | R2-160_H159P_P207S |
| 205 | R2-161_H159P_P207Q |
| 206 | R2-162_H159P_P207A |
| 207 | R2-163_H159P_P207G |
| 208 | R2-164_Q127Y_H159P_P207N |
| 209 | R2-165_Q127Y_H159P_P207S |
| 210 | R2-166_Q127Y_H159P_P207Q |
| 211 | R2-167_Q127Y_H159P_P207A |
| 212 | R2-168_Q127Y_H159P_P207G |
| 213 | R2-169_Q127M_H159P_P207N |
| 214 | R2-170_Q127M_H159P_P207S |
| 215 | R2-171_Q127M_H159P_P207Q |
| 216 | R2-172_Q127M_H159P_P207A |
| 217 | R2-173_Q127M_H159P_P207G |
| 218 | R2-174_Q127Y_H159E_P207N |
| 219 | R2-175_Q127Y_H159E_P207S |
| 220 | R2-176_Q127Y_H159E_P207Q |
| 221 | R2-177_Q127Y_H159E_P207A |
| 222 | R2-178_Q127Y_H159E_P207G |
| 223 | R2-179_Q127M_H159E_P207N |
| 224 | R2-180_Q127M_H159E_P207S |
| 225 | R2-181_Q127M_H159E_P207Q |
| 226 | R2-182_Q127M_H159E_P207A |
| 227 | R2-183_Q127M_H159E_P207G |
| 228 | R2-184_Q127H_H159V_P207N |
| 229 | R2-185_Q127H_H159V_P207S |
| 230 | R2-186_Q127H_H159V_P207Q |
| 231 | R2-187_Q127H_H159V_P207A |
| 232 | R2-188_Q127H_H159V_P207G |
| 233 | R2-189_Q127E_H159A_P207N |
| 234 | R2-190_Q127E_H159A_P207S |
| 235 | R2-191_Q127E_H159A_P207Q |
| 236 | R2-192_Q127E_H159A_P207A |
| 237 | R2-193_Q127E_H159A_P207G |

As a first step, the initial panel of 95 cell lines expressing ortho-IL2RG variants carrying single substitutions (Table 1) was retested with a new panel of CV415 variants. The cells were exposed to two concentrations (100 and 17 ng/ml) of each of the 18 variants of CV415 or control cytokines (namely, CV415, wild-type IL-21, and a form of wild-type IL-21 carrying the Q116K substitution (SEQ ID NO: 142). Singlet determinations were made in each case, such that two concentrations of two cytokines could be tested against the 96 cell lines using a single 384-well plate (as before, the panel of cells expressing 95 IL2RG variants was supplemented with a control cell line expressing wild-type human IL2RG).

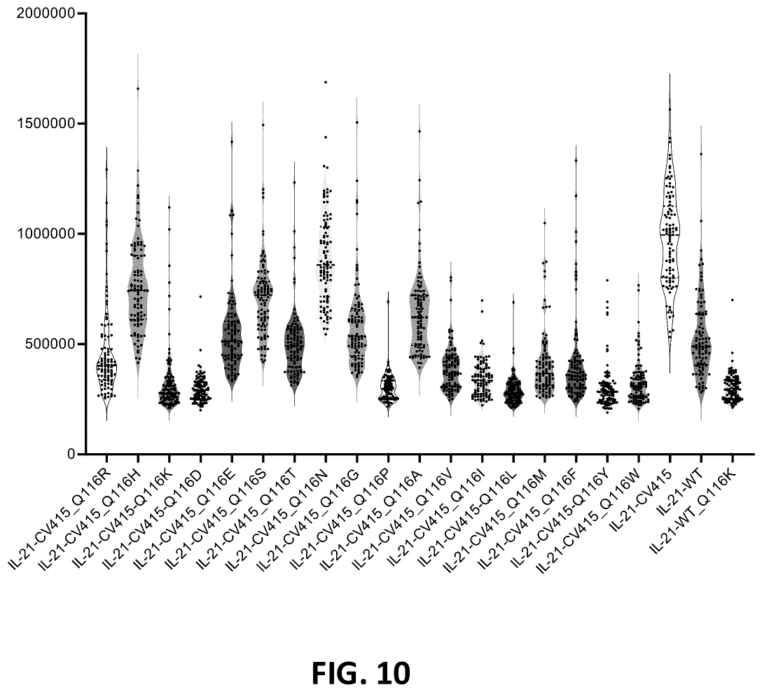

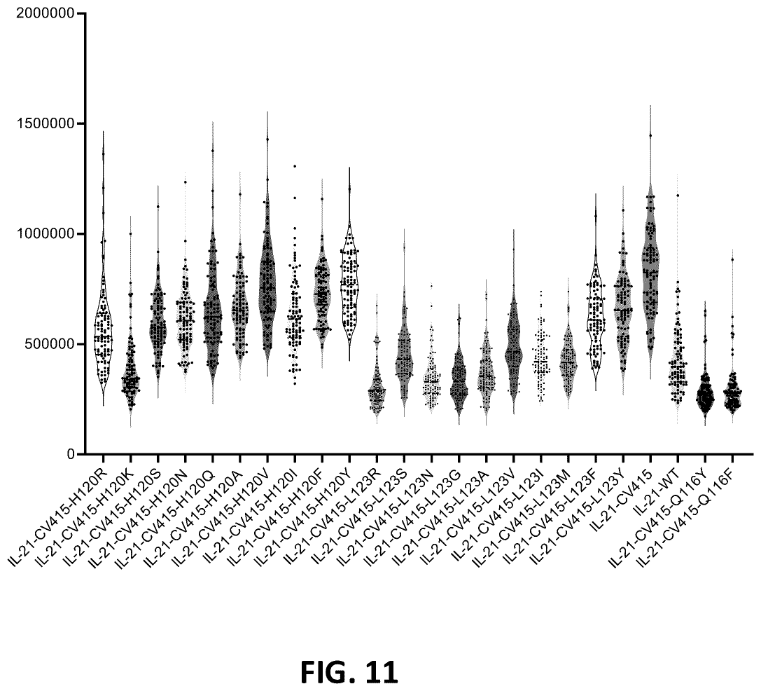

The box-violin plots in FIGS. 10 and 11 show the responses made by the 96 cell lines to each of the indicated cytokines at 17 ng/ml. This global perspective on the data makes clear that among substitutions at residue 116 (FIG. 10), Q116K, Q116D, Q116P, Q116L, Q116Y, and Q116W severely compromised the capacity of CV415 to induce responses in the Ba/F3 cells for the majority of IL2RG variants. By contrast, multiple of the other Q116 substitutions demonstrated distinct patterns of reactivity with the panel of cytokines. Q116N, for example, showed a broad reactivity distribution that grossly resembled the distribution associated with CV415. Q116H, Q116E, Q116S, Q116T, Q116G, and Q116A generated distributions with intermediate mean responses. Importantly, all the cell lines showed strong reactivity against the parent CV415 cytokine, which was expected because the Ba/F3 cells retain expression of the endogenous mouse IL2RG molecule.

Among ten substitutions at position 120, H120K had the most severe impact on reactivity when tested against the panel of cell lines (FIG. 11); most of the other substitutions at this position had relatively modest effects on the reactivity distributions. By contrast, most of the substitutions at position 123 impaired reactivity more severely (FIG. 11).

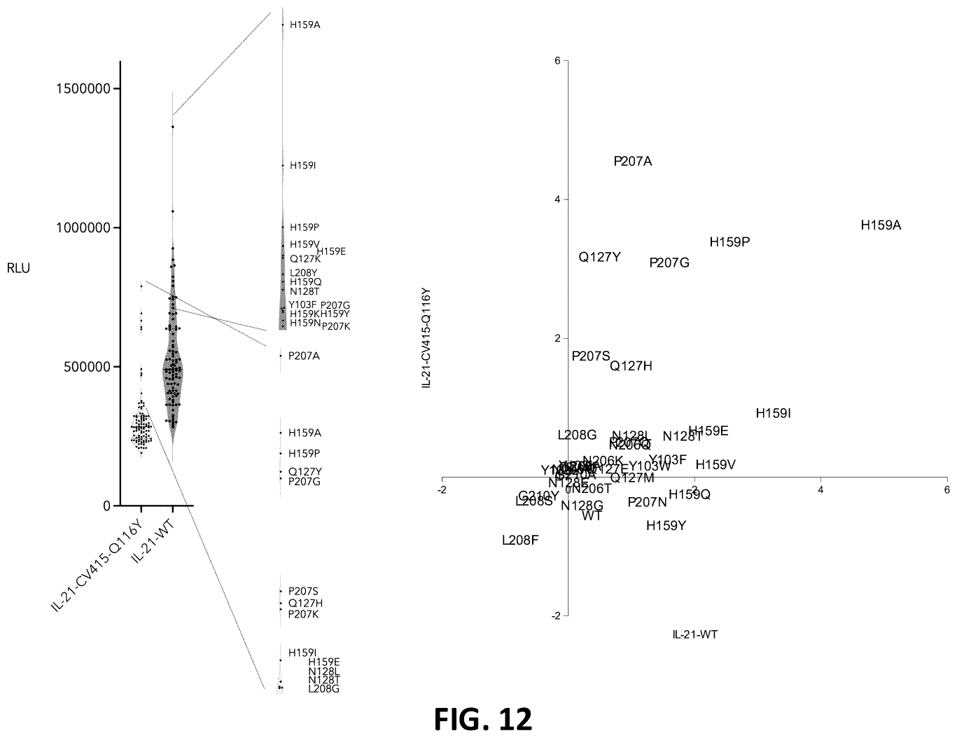

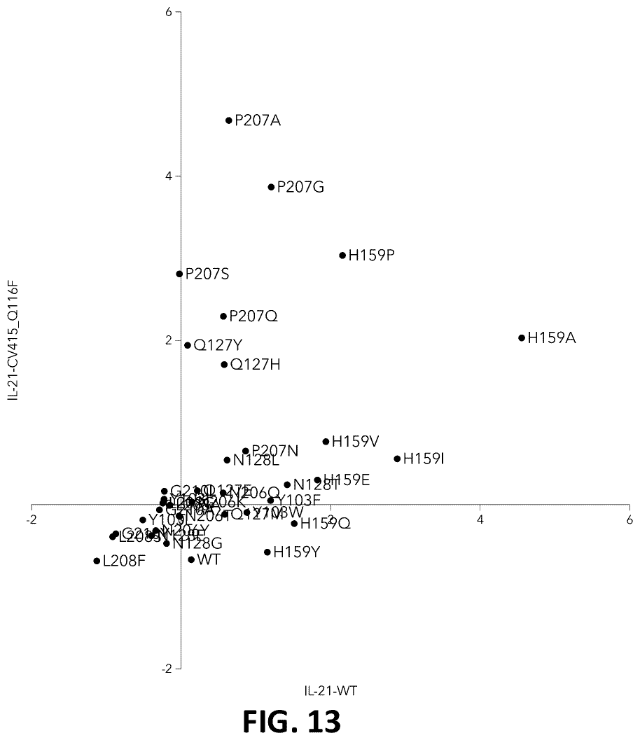

FIG. 12 provides detail concerning the identity of ortho-IL2RG candidates found at the top of the Q116Y and IL-21-WT distributions from FIG. 10. The distributions in FIG. 10 were transformed into Z-scores to facilitate comparisons between them. A subset of the Z-scores (comprised of a common collection of IL2RG variants) was used to generate bivariate plots of the sort shown in the righthand panel of FIG. 12. This kind of plot was useful in discriminating IL2RG substitutions associated with increased reactivity for specific CV415 variants. The data in FIG. 12 identify P207A, Q127Y, P207G, P207S, and Q127H as substitutions that enhance reactivity for CV415-Q116Y. FIG. 13 affords similar observations for residues that enhance reactivity for CV415-Q116F. These two cytokines (CV415-Q116Y and CV415-Q116F) were of interest for further development because of the strong effects shown in FIGS. 12 and 13.

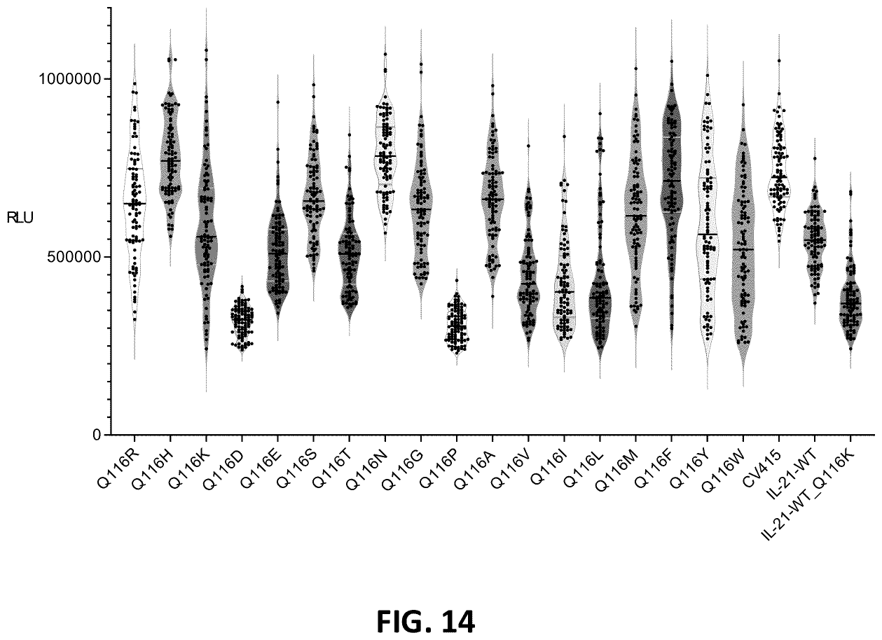

The panel of CV415 variants carrying Q116 substitutions was retested for reactivity against cell lines carrying IL2RG variants with double or triple substitutions (Table 4). A similar experimental design was employed to the one used for FIGS. 10 and 11, and the results obtained (for cytokines used at 17 ng/ml) are shown in FIG. 14. As before, the Q116D and Q116P substitutions severely impaired responses to the entire panel of cell lines. Strikingly, however, multiple other substitutions (such as Q116Y or Q116F) that were also mostly disabling when used with the prior cell line panel (FIG. 10) were much less impactful when used with the second cell line panel. The new panel is therefore enriched with IL2RG variants that have improved capacity to induce responses in cells in response cytokines carrying specific Q116 substitutions.

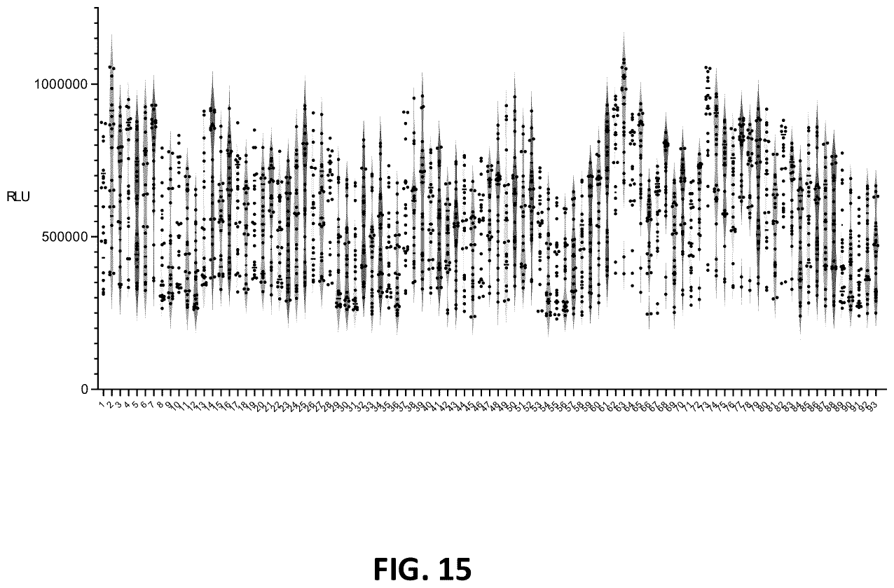

FIG. 15 provides an alternative perspective on the same dataset presented in FIG. 14. This representation of the data identifies some ortho-IL2RG candidates that potentiate responses to multiple ortho-IL-21 candidates (such as variants R2-102, R2-163, and R2-173), and others that are generally less active (such as variants R2-112, R2-155, and R2-191).

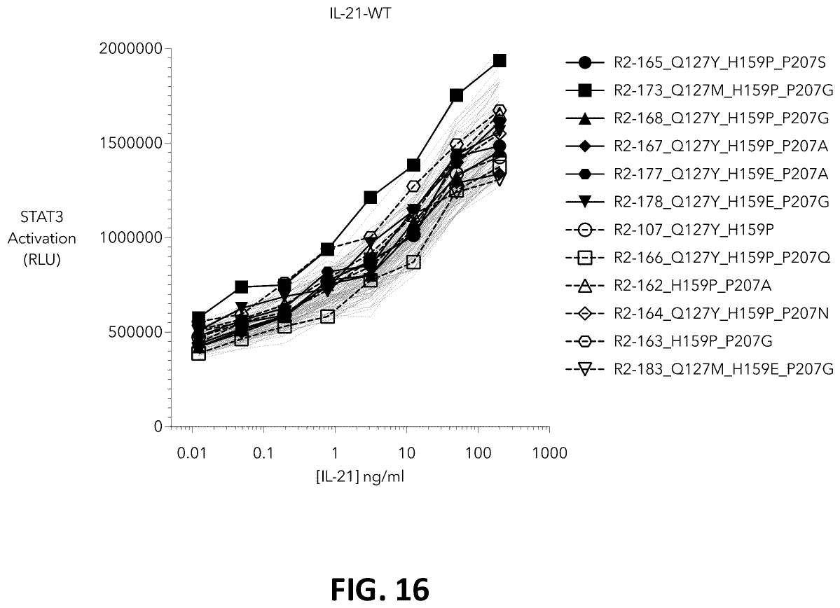

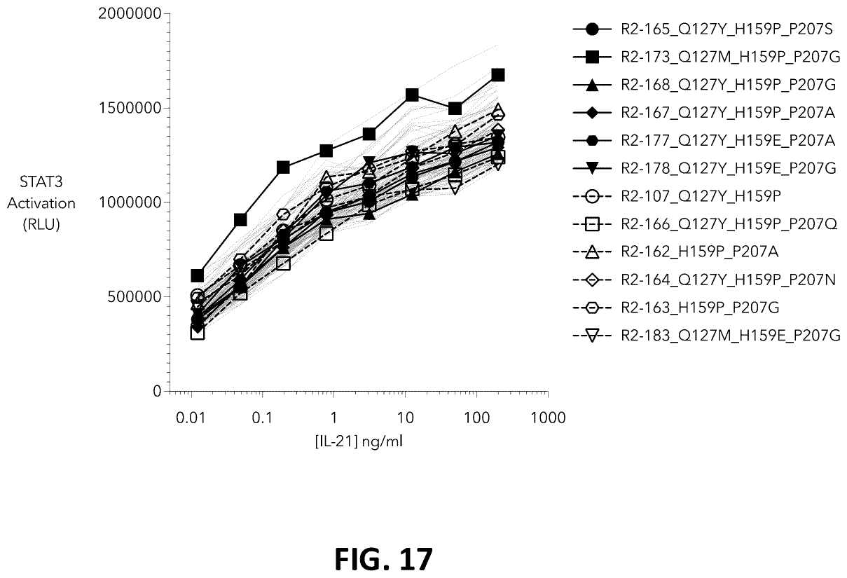

An additional experiment was performed in which the full panel of cell lines carrying double or triple substitutions was tested against titrations of selected cytokines (FIGS. 16-19). As before, the Ba/F3 cells used in these experiments retained expression of endogenous CD132, accounting for their capacity to make responses to wild-type IL-21 (FIG. 16) and CV415 (FIG. 17). The latter responses were stronger than the former, as expected. The response curves for the majority of cell lines were clustered in both cases, with only a minority (including the one for variant R2-173) being distinctive.

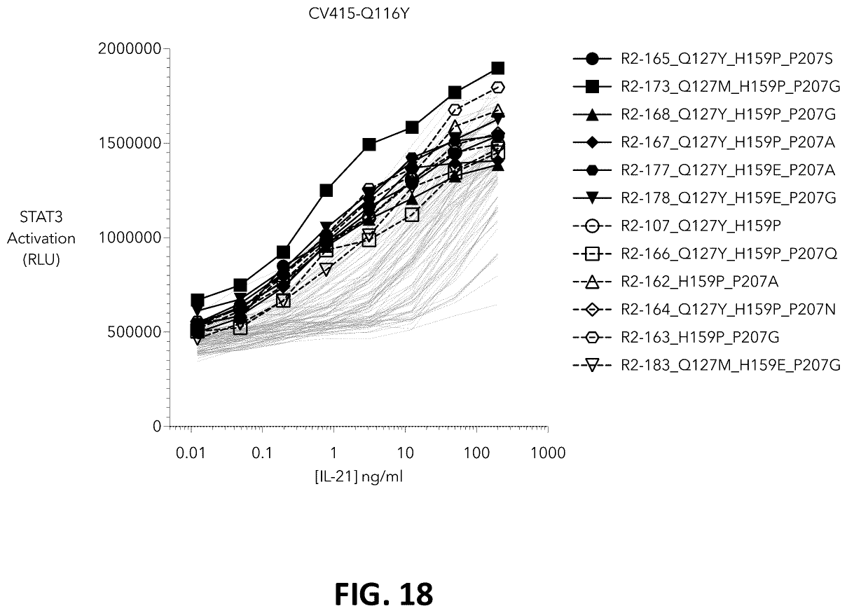

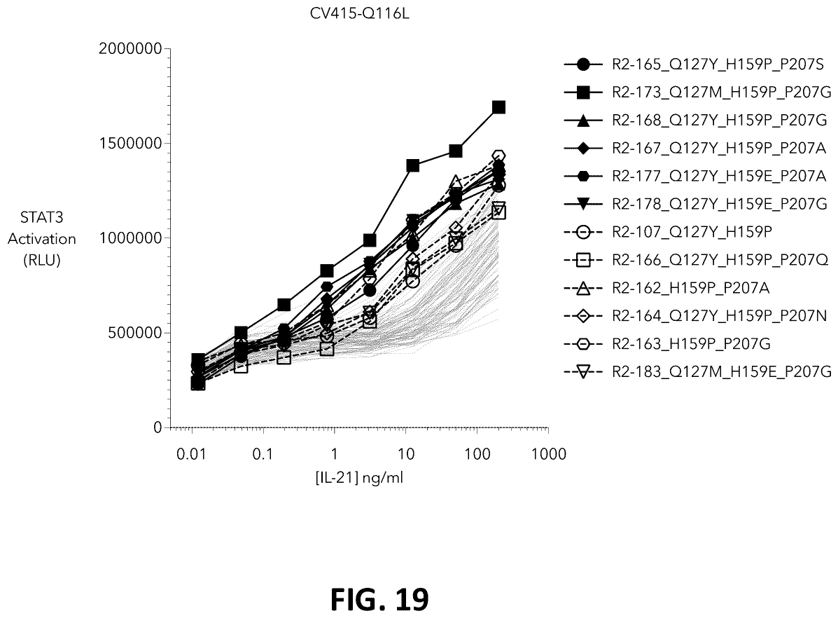

As shown in FIGS. 18 and 19, a quite different distribution of response curves was obtained when the CV415 cytokine carried a Q116Y or Q116L substitution. These cytokines have very much attenuated activity when cells express only the endogenous mouse form of CD132 (FIG. 7). The strong responses elicited by the cytokines in multiple cell lines can therefore be attributed to the variant IL2RG molecules they express.

A further collection of IL2RG variants was generated based on four of the variants (R2-168, R2-173, R2-177, and R2-182) that were associated with good responses to CV415 carrying Q116 substitutions. Twenty-four new IL2RG variants (Table 5) were generated for each of these four founder variants (for a total of 96 variants) using substitutions enriched with those that might confer enhanced responses to CV415 carrying Q116, H120, or L123 substitutions based on data of the sort shown in FIGS. 12 and 13.

TABLE 5

| SEQ ID NO: | ortho-IL2RG candidate_Substitution |
|---|---|
| 238 | R3-201_168_Y103L |
| 239 | R3-202_168_Y103F |
| 240 | R3-203_168_Y103W |
| 241 | R3-204_168_Y103I |
| 242 | R3-205_168_N128E |
| 243 | R3-206_168_N128T |
| 244 | R3-207_168_N128G |
| 245 | R3-208_168_N128L |
| 246 | R3-209_168_N206K |
| 247 | R3-210_168_N206Y |
| 248 | R3-211_168_N206Q |
| 249 | R3-212_168_N206T |
| 250 | R3-213_168_L208S |
| 251 | R3-214_168_L208A |
| 252 | R3-215_168_L208F |
| 253 | R3-216_168_L208G |
| 254 | R3-217_168_G210I |
| 255 | R3-218_168_G210A |
| 256 | R3-219_168_G210Y |
| 257 | R3-220_168_N128G_N206K |
| 258 | R3-221_168_N128G_L208S |
| 259 | R3-222_168_N128G_Y103L |
| 260 | R3-223_168_N128G_G210A |
| 261 | R3-224_168_N128G_N206K_Y103L |
| 262 | R3-225_173_Y103L |
| 263 | R3-226_173_Y103F |
| 264 | R3-227_173_Y103W |
| 265 | R3-228_173_Y103I |
| 266 | R3-229_173_N128E |
| 267 | R3-230_173_N128T |
| 268 | R3-231_173_N128G |
| 269 | R3-232_173_N128L |
| 270 | R3-233_173_N206K |
| 271 | R3-234_173_N206Y |
| 272 | R3-235_173_N206Q |
| 273 | R3-236_173_N206T |
| 274 | R3-237_173_L208S |
| 275 | R3-238_173_L208A |
| 276 | R3-239_173_L208F |
| 277 | R3-240_173_L208G |
| 278 | R3-241_173_G210I |
| 279 | R3-242_173_G210A |
| 280 | R3-243_173_G210Y |
| 281 | R3-244_173_N128G_N206K |
| 282 | R3-245_173_N128G_L208S |
| 283 | R3-246_173_N128G_Y103L |
| 284 | R3-247_173_N128G_G210A |
| 285 | R3-248_173_N128G_N206K_Y103L |
| 286 | R3-249_177_Y103L |
| 287 | R3-250_177_Y103F |
| 288 | R3-251_177_Y103W |
| 289 | R3-252_177_Y103I |
| 290 | R3-253_177_N128E |
| 291 | R3-254_177_N128T |
| 292 | R3-255_177_N128G |
| 293 | R3-256_177_N128L |
| 294 | R3-257_177_N206K |
| 295 | R3-258_177_N206Y |
| 296 | R3-259_177_N206Q |
| 297 | R3-260_177_N206T |
| 298 | R3-261_177_L208S |
| 299 | R3-262_177_L208A |
| 300 | R3-263_177_L208F |
| 301 | R3-264_177_L208G |
| 302 | R3-265_177_G210I |
| 303 | R3-266_177_G210A |
| 304 | R3-267_177_G210Y |
| 305 | R3-268_177_N128G_N206K |
| 306 | R3-269_177_N128G_L208S |
| 307 | R3-270_177_N128G_Y103L |
| 308 | R3-271_177_N128G_G210A |
| 309 | R3-272_177_N128G_N206K_Y103L |
| 310 | R3-273_182_Y103L |
| 311 | R3-274_182_Y103F |
| 312 | R3-275_182_Y103W |
| 313 | R3-276_182_Y103I |
| 314 | R3-277_182_N128E |
| 315 | R3-278_182_N128T |
| 316 | R3-279_182_N128G |
| 317 | R3-280_182_N128L |
| 318 | R3-281_182_N206K |
| 319 | R3-282_182_N206Y |
| 320 | R3-283_182_N206Q |
| 321 | R3-284_182_N206T |
| 322 | R3-285_182_L208S |
| 323 | R3-286_182_L208A |
| 324 | R3-287_182_L208F |
| 325 | R3-288_182_L208G |
| 326 | R3-289_182_G210I |
| 327 | R3-290_182_G210A |
| 328 | R3-291_182_G210Y |
| 329 | R3-292_182_N128G_N206K |
| 330 | R3-293_182_N128G_L208S |
| 331 | R3-294_182_N128G_Y103L |
| 332 | R3-295_182_N128G_G210A |
| 333 | R3-296_182_N128G_N206K_Y103L |

Twelve additional cytokines were also generated carrying combinations of tyrosine or phenylalanine substitutions at Q116, H120, and L123 (Table 6).

TABLE 6

| SEQ ID NO | ortho-IL-21 candidate |
|---|---|
| 334 | CV415_Q116Y_H120F |
| 335 | CV415_Q116Y_H120F_L123F |
| 336 | CV415_Q116Y_H120F_L123Y |
| 337 | CV415_Q116Y_H120Y |
| 338 | CV415_Q116Y_H120Y_L123F |
| 339 | CV415_Q116Y_H120Y_L123Y |
| 340 | CV415_Q116F_H120F |
| 341 | CV415_Q116F_H120F_L123F |
| 342 | CV415_Q116F_H120F_L123Y |
| 343 | CV415_Q116F_H120Y |
| 344 | CV415_Q116F_H120Y_L123F |
| 345 | CV415_Q116F_H120Y_L123Y |

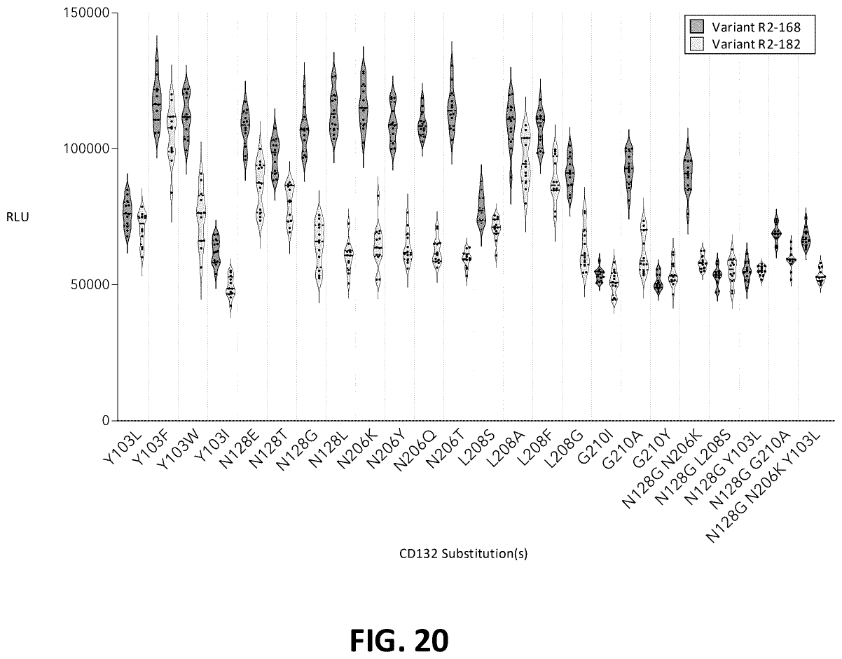

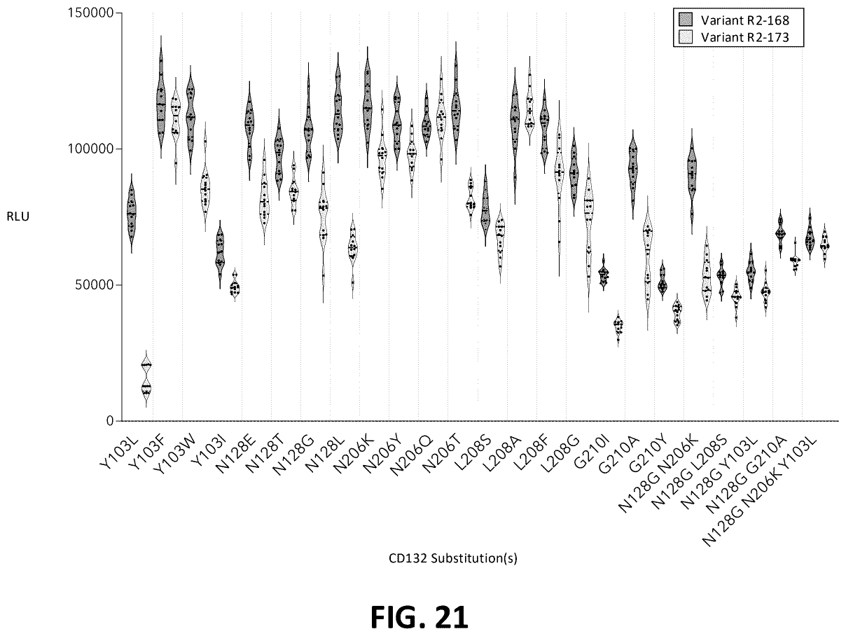

Ninety-six Ba/F3 cell lines expressing both RV22 and the new IL2RG variants were generated as before using sequential puromycin and hygromycin selection regimens. The cells were then tested for reactivity against two concentrations (0.1 and 1 ng/ml) of the 12 new cytokines together with various control cytokines. A representation of a subset of the results obtained is provided in FIGS. 20-22. These figures group the IL2RG variants according to their origin (i.e., whether they were based on R2-168, R2-173, R2-177, or R2-182) allowing for an assessment of whether any of the compounded substitutions might differ in how they impact responses to the cytokine collection as a function of their origin. This type of difference is evident in all three figures. As an example, whereas Y103F had a similar effect on the four founding IL2RG variants (R2-168, R2-173, R2-177, and R2-182), N206T and N126L were more deleterious to responses made by R2-173, R2-177 and R2-182 than to R2-168.

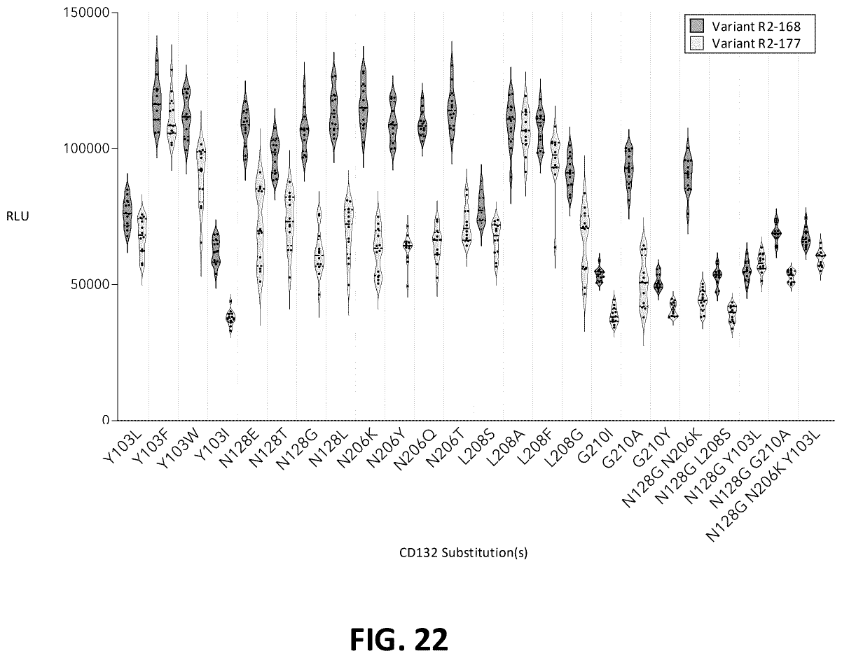

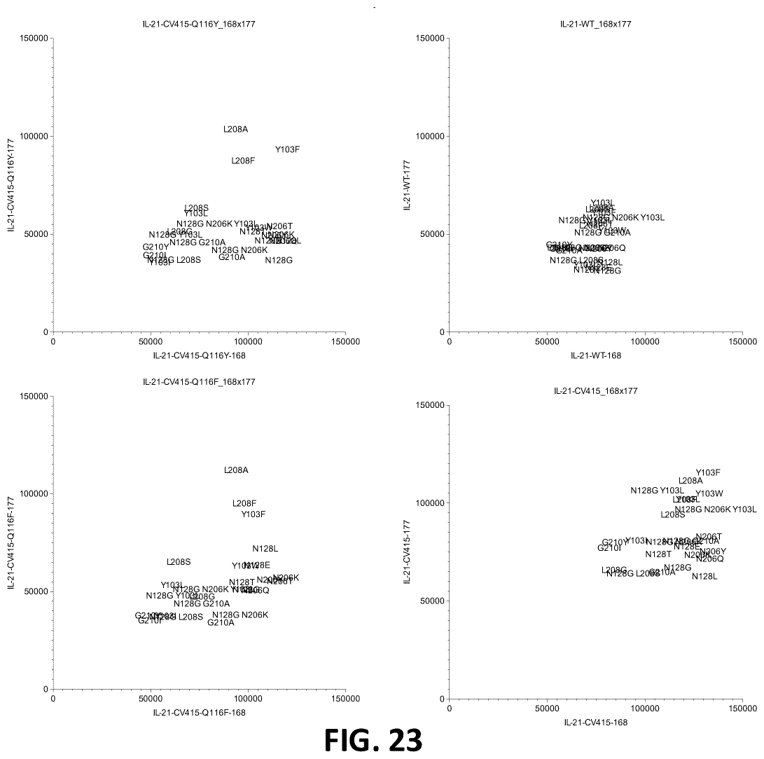

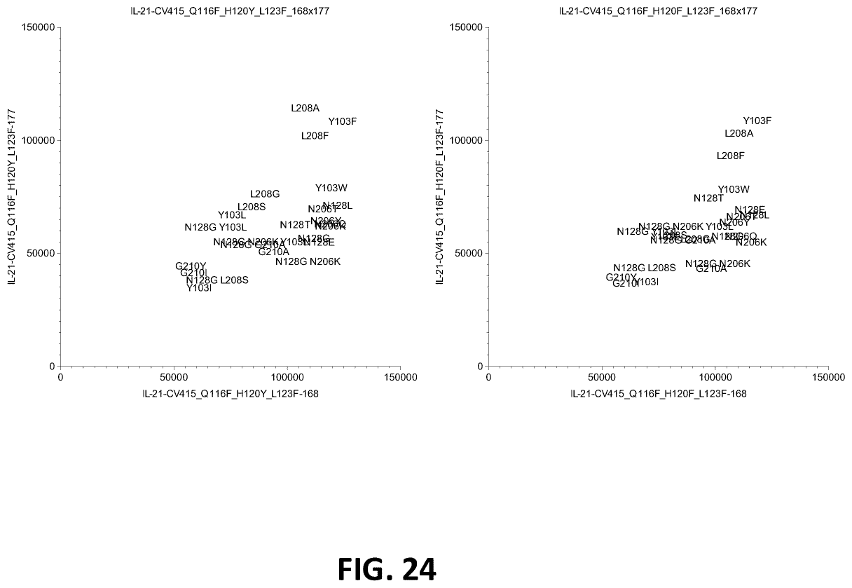

The responses made by the four cell line collections (i.e., those based on R2-168, R2-173, R2-177, or R2-182) to different cytokines were compared using bivariate plots of the sort shown in FIGS. 23 and 24. These plots show that substitutions such as L208A, L208F and Y103F are associated with strong responses to CV415 bearing the Q116Y or Q116F substitutions regardless of whether the R2-168 or R2-177 IL2RG variant is used. By contrast, N206K or N128E potentiate responses by R2-168 preferentially (as is also apparent in FIG. 22).

Twenty-one IL2RG variants were selected for further analysis based on the results just summarized. Cells expressing these variants (and three control cell lines expressing RV22, RV34 (SEQ ID NO: 354), or wild-type IL-21Rα in association with the endogenous mouse form of CD132) were tested for their responses to a titration of the variant and control cytokines.

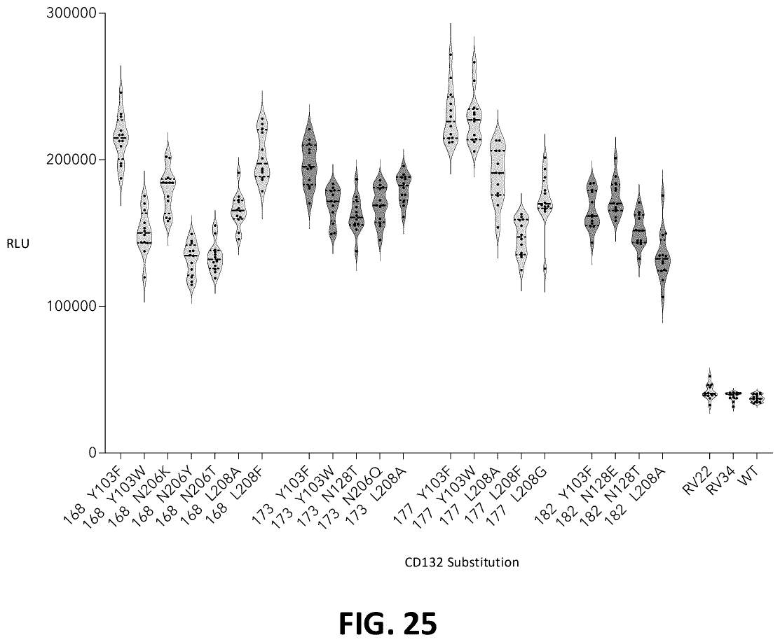

FIG. 25 shows the responses made by the cell lines to the 12 cytokine variants listed in Table 6 at a single concentration (8 ng/ml). This figure makes clear that the variant cytokines selectively elicit responses in cells expressing all the IL2RG variants but not in cells expressing endogenous CD132. It also shows that all twelve of the variant cytokines have the capacity to induce responses in cells bearing the IL2RG variants.

To test further the specificity of the variant forms of IL2RG and to assess the extent to which they might provide a general enhancement of responsiveness, a panel of cell lines was generated that expressed the wild-type form of IL-21Rα together with the same twenty-one IL2RG variants represented in FIG. 25. These cells (and their RV22-expressing counterparts as in.

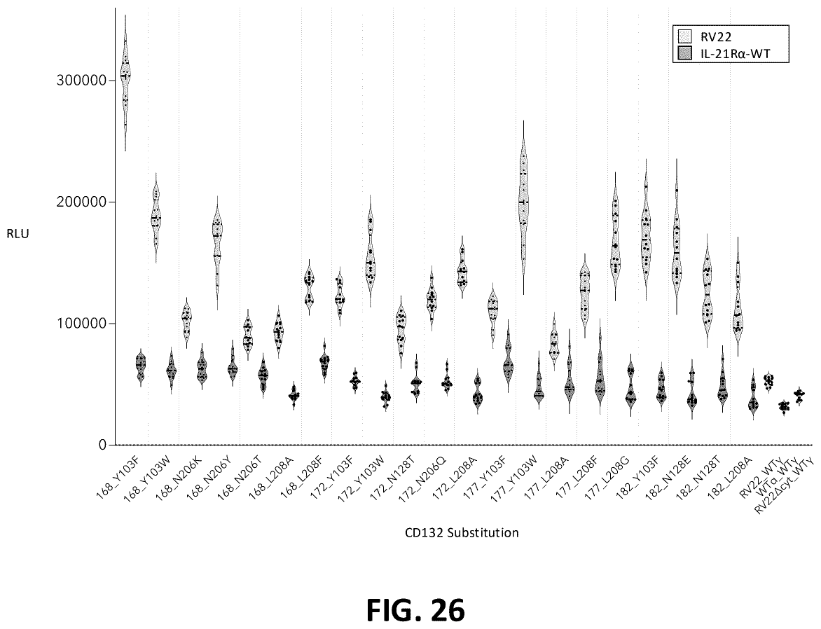

FIG. 25) were then tested for their capacity to respond to a titration of variant and control cytokines. FIG. 26 shows the responses made by the panel of cells to the cytokines listed in Table 6 at a single concentration (4 ng/ml). The results make clear that in most cases, the potentiation afforded by the IL2RG variants is restricted to cells expressing the cognate IL-21Rα chain (RV22) with much weaker effects evident on responses involving the wild-type form of IL-21Rα. The IL2RG variants do not therefore enhance responses in a fashion that would overcome the effect of cytokine substitutions that impair binding to IL-21Rα (i.e., the substitutions present in CV415 that account for selective binding to RV22 over IL-21Rα-WT).

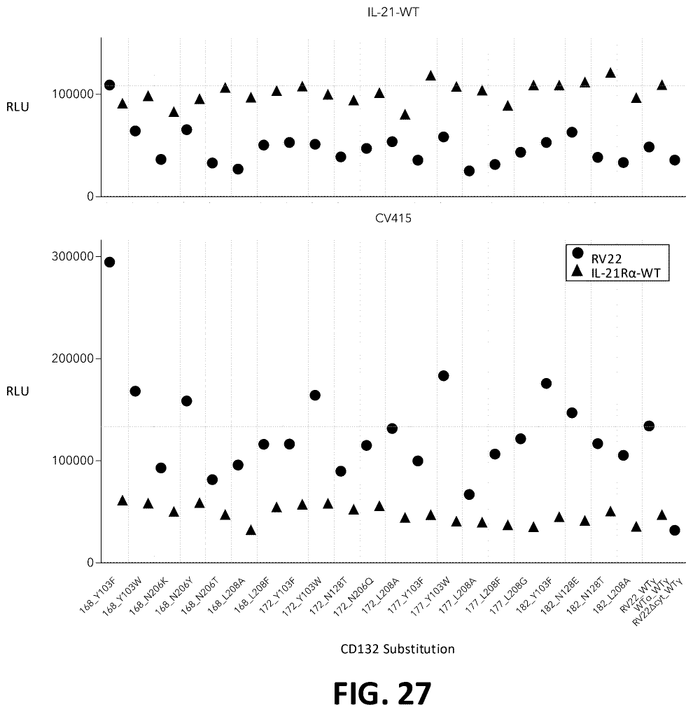

Similar observations derived from a comparison of responses made by the cell panels to the wild-type form of IL-21 versus CV415 (without Q116, H120 or L123 substitutions). As expected, cells expressing wild-type IL-21Rα generally responded more strongly to the former than to the latter (the top panel of FIG. 27 shows these responses measured when the cytokines were used at 4 ng/ml). These responses are likely to be partially, if not entirely, dependent on the involvement of endogenous mouse CD132. Comparisons to the three control cell lines-featuring ectopic expression of wild-type human IL2RG (at the extreme right of the figure)—show there was minimal or no enhancement of responsiveness to wild-type IL-21 due to the presence of the variant IL2RG molecules.

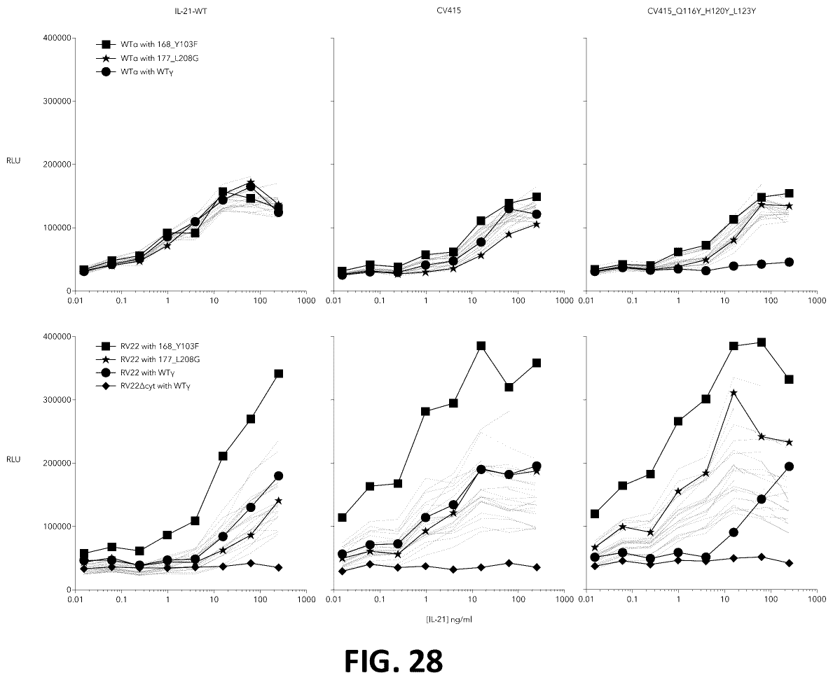

The bottom panel of FIG. 27 shows a more variable effect of the IL2RG variants on responses made to CV415 by cells expressing RV22, but not by cells expressing the wild-type form of IL-21Rα. FIG. 28 provides a similar perspective: cells expressing the wild-type form of IL-21Rα (top respond panel) similarly to IL-21-WT, CV415, and CV415_Q116Y_H120Y_L123Y regardless of the IL2RG variant they express, but there is considerable variability in the response curves made by cells expressing RV22 (bottom panel) and the same IL2RG variants.

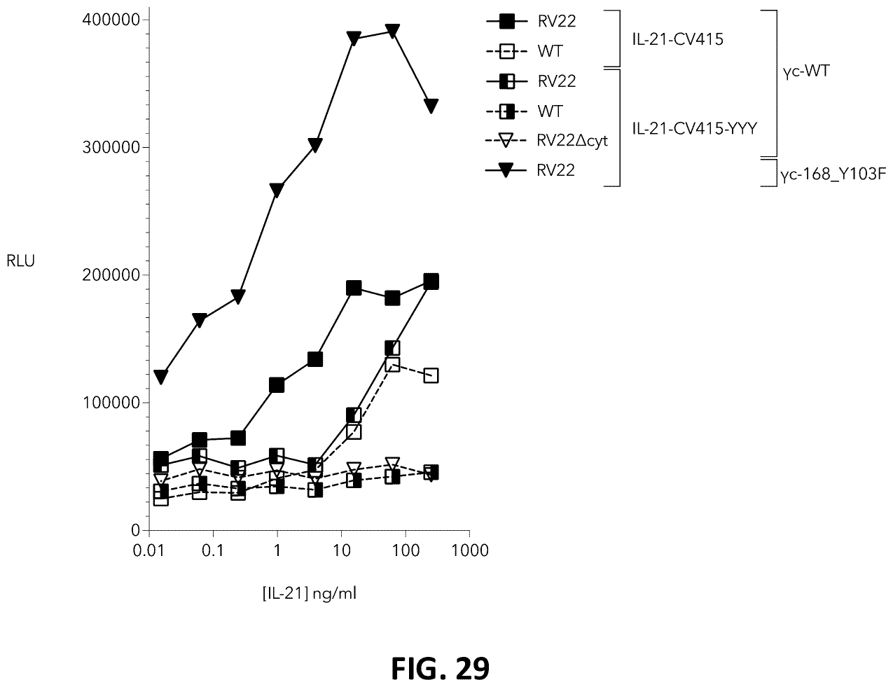

The observations just summarized show that the IL2RG variants potentiate responses made by cells expressing RV22, and in some cases, such as with variant 177_L208G, this potentiation only occurs with CV415 carrying a substitution (such as CV415_Q116Y_H120Y_L123Y in FIG. 28). Crucially, this same cytokine is inactive when used with cells expressing the wild-type form of IL-21Rα with wild-type IL2RG (FIG. 28, top right). Thus, the combination of RV22, IL2RG-177_L208G, and CV415_Q116Y_H120Y_L123Y create a cytokine-receptor system that is orthogonal to that of the natural IL-21R-IL-21 system. The same sort of conclusion can be drawn from the results in FIG. 29, which shows a strong response to CV415_Q116Y_H120Y_L123Y made by cells expressing RV22 in combination with IL2RG-168_Y103F. Here again, this same cytokine fails to elicit a response in cells expressing the wild-type IL-21 receptor.

The ortho-IL-21/ortho-IL-21Rα base system, that is, CV415/RV22, was disclosed and enabled in U.S. Nonprovisional patent application Ser. No. 18/304,172, and was chosen for use here because of the potential for the orthogonal IL2RG interaction to improve the performance of the system. However, any of the ortho-IL-21/ortho-IL-21Rα systems disclosed and enabled in U.S. Nonprovisional patent application Ser. No. 18/304,172 could have been used with an equally good expectation of success and, along with the additional amino acid substitutions at positions Q116, H120, and L123 of the IL-21 peptide, should be considered within the scope of the instant invention as if fully set forth herein.

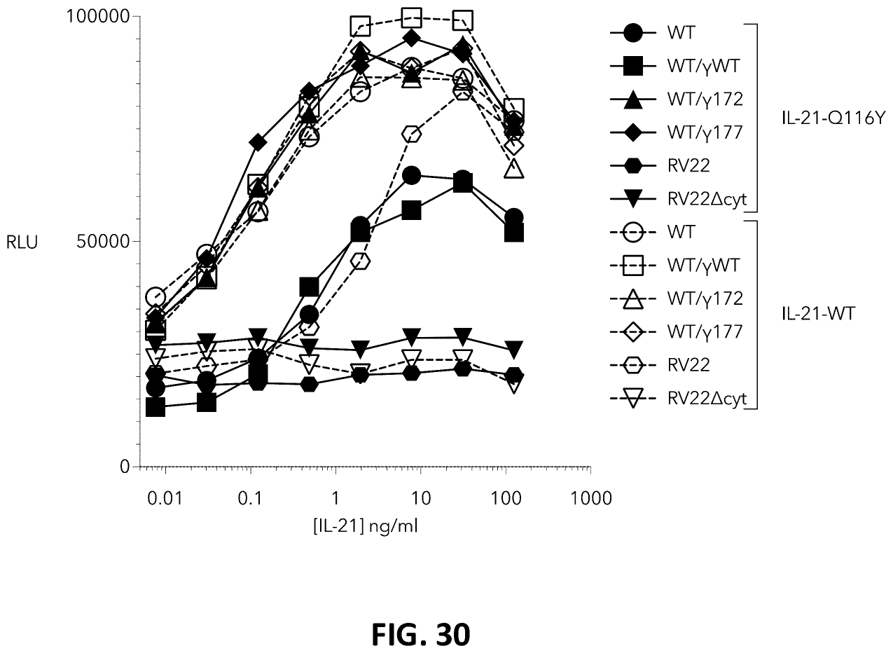

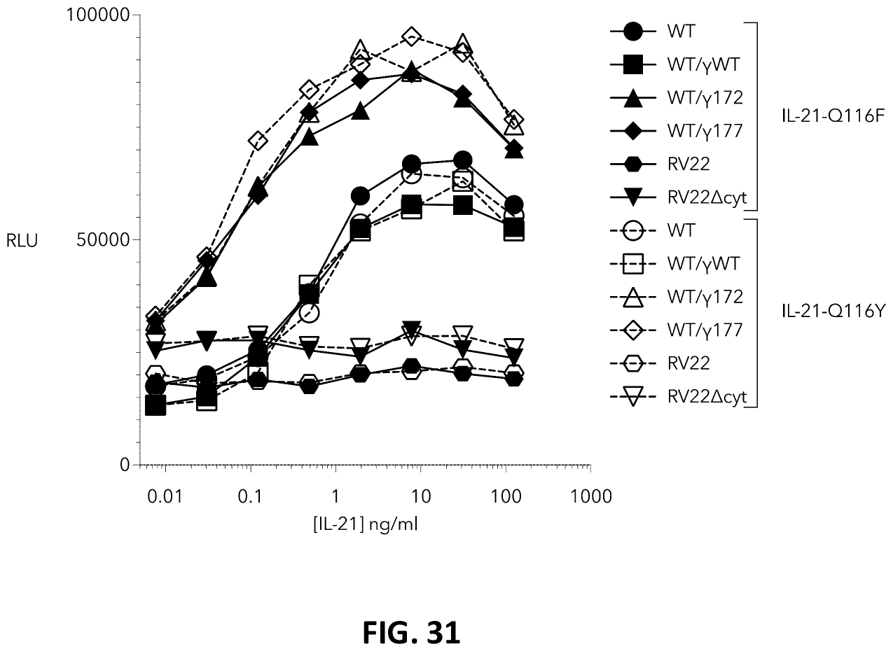

To test whether an orthogonal cytokine-receptor system could be developed based solely on variation in the gamma chain, a form of IL-21 was generated carrying the Q116Y and no other substitutions (IL-21-Q116Y, SEQ ID NO 143). As expected, this cytokine elicited impaired responses in cells expressing the wild-type form of IL-21Rα relative to responses elicited by wild-type IL-21 (FIG. 30). Strikingly, it induced no responses in cells expressing RV22. A similar outcome was obtained when a form of IL-21 carrying the Q116F substitution was used instead of the Q116Y variant (SEQ ID NO 144; FIG. 31). Expression of the gamma chain variants R2-172 or R2-177 restored the responses to equivalency with the fully wild-type situation. It is possible that additional substitutions in IL-21 that affect the IL2RG interaction (such as those at residues 120 or 123) may further impair binding of the modified IL-21 to the wild-type receptor. Similarly, additional variation in IL2RG may enhance binding to such variants. The results of this experiment, therefore, suggest that it may be possible to construct an orthogonal cytokine-receptor system based solely on variation in IL2RG.

IL-2 and IL-21 engage with CD132 in a very similar fashion with glutamine-126 in the former being analogous to glutamine-116 in the latter (Abhiraman, G. C. et al. A structural blueprint for interleukin-21 signal modulation. Cell Rep. 2023; 42 (6): 112657). It is likely, therefore, that IL2RG variants described here, or based on those described here, may perform a similar response-potentiating function when used with forms of IL-2 carrying substitutions at Q126. An orthogonal system based on IL-2 would be of interest for cell therapy because of the potency with which this cytokine enhances T cell responses. Orthogonal systems based on IL-4, IL-7, IL-9, and IL-15 may also be constructed using related approaches employing derivative or functionally analogous forms of IL2RG. A particularly appealing aspect of such systems is the potential they may provide for improving the safety of cellular therapies by rendering T cells exclusively dependent on an orthogonal gamma cytokine for their proliferation and survival.

1. An engineered gamma chain, CD132, comprising amino acid substitutions, numbered relative to SEQ ID NO: 348, of:
Q127Y or Q127M;
H159P or H159E; and
P207G or P207A.

2. The engineered gamma chain of claim 1, comprising amino acid substitutions Q127Y/H159P/P207G.

3. The engineered gamma chain of claim 1, comprising amino acid substitutions Q127M/H159P/P207G.

4. The engineered gamma chain of claim 1, comprising amino acid substitutions Q127Y/H159E/P207A.

5. The engineered gamma chain of claim 1, comprising amino acid substitutions Q127M/H159E/P207A.

6. The engineered gamma chain of claim 1, further comprising an amino acid substitution at position Y103 selected from the group consisting of Y103L, Y103F, Y103W, and Y103I.

7. The engineered gamma chain of claim 1, further comprising an amino acid substitution at position N128 selected from the group consisting of N128E, N128T, N128G, and N128L.

8. The engineered gamma chain of claim 1, further comprising an amino acid substitution at position N206 selected from the group consisting of N206K, N206Y, N206Q, and N206T.

9. The engineered gamma chain of claim 1, further comprising an amino acid substitution at position L208 selected from the group consisting of L208S, L208A, L208F, and L208G.

10. The engineered gamma chain of claim 1, further comprising an amino acid substitution at position G210 selected from the group consisting of G210I, G210A, and G210Y.

11. A modified cell expressing the engineered gamma chain of claim 1.

12-26. (canceled)

27. The modified cell of claim 11, wherein the modified cell is selected from the group consisting of a lymphocyte, an engineered lymphocyte, and an engineered lymphocyte comprising a T-cell engineered to express a chimeric antigen receptor.

28-30. (canceled)

31. The engineered human IL-21 receptor of claim 36, wherein the native cytoplasmic domain of the alpha chain of the engineered human IL-21 receptor has been replaced with the cytoplasmic domain from one of: IL-2Rβ (CD122), IL-4Rα (CD124), IL-7Rα (CD127), IL-9Rα (CD129), IL10RA (CD210), or IL10RB (CDW210B).

32. A modified cell expressing the engineered human IL-21 receptor of claim 36.

33-35. (canceled)

36. An engineered human IL-21 receptor having a native alpha chain, CD360, and an engineered gamma chain, CD132, the gamma chain comprising amino acid substitutions, numbered relative to SEQ ID NO: 348, of:
Q127Y or Q127M;
H159P or H159E; and
P207G or P207A.

37-38. (canceled)

39. A pharmaceutical composition comprising the modified cell of claim 27.

40. A polynucleotide encoding for the engineered gamma chain of claim 1.

41. A pharmaceutical composition comprising the modified cell of claim 32.

42. A polynucleotide encoding for the engineered gamma chain of claim 36.

43. A modified cell expressing the engineered human IL-21 receptor of claim 31.


SEQUENCE LISTING (355 sequences)

SEQ ID NO: 1 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLFQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLWQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 3 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLMQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 4 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLIQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 6 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLQQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 7 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLAQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 8 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLPQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLGQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 10 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLHQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLNNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 12 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 13 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLKNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 14 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 15 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLRNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 16 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLDNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 17 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLSNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 18 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 20 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLGNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 21 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLPNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 22 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQLLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 23 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQELVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 24 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQRLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 25 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQSLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 26 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQTLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 27 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQKLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 28 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQHLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 29 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQGLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 30 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQALVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 31 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQDLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 32 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQQLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 33 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQPLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 34 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 35 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNKCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 36 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNRCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 37 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 38 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 39 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNNCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 40 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 41 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 42 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 43 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 44 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFSPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 45 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFAPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 46 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFHPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 47 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFTPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 48 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFGPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 49 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFEPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 50 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFDPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 51 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFIPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 52 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFQPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 53 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFYPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 54 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFKPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 55 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFPPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 56 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 57 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 58 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNELCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 59 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNKLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 60 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNTLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 61 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 62 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNILCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 63 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 64 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 65 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNVLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 66 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPYCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 67 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPFCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 68 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPSCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 69 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPVCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 70 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPNCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 71 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPRCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 72 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPICGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 73 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPACGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 74 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPGCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 75 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPPCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 76 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 77 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCISAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 78 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCLSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 79 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCMSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 80 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCTSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 81 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCSSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 82 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCYSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 83 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCWSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 84 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCFSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 85 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCVSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 86 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGNAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 87 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGTAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 88 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGRAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 89 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGMAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 90 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGIAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 91 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGGAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 92 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGYAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 93 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGWAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 94 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGFAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 95 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGVAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 96 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHLSSRTHGSEDS

SEQ ID NO: 97 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLDKMIHQHLSSRTHGSEDS

SEQ ID NO: 98 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLDKMIHQHDSSRTHGSEDS

SEQ ID NO: 99 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLDKMIDQHLSSRTHGSEDS

SEQ ID NO: 100 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIHQHLSSRTHGSEDS

SEQ ID NO: 101 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIHQHDSSRTHGSEDS

SEQ ID NO: 102 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIDQHLSSRTHGSEDS

SEQ ID NO: 103 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLKKMIHQHLSSRTHGSEDS

SEQ ID NO: 104 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLKKMIHQHDSSRTHGSEDS

SEQ ID NO: 105 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLKKMIDQHLSSRTHGSEDS

SEQ ID NO: 106 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLLKMIHQHLSSRTHGSEDS

SEQ ID NO: 107 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLLKMIHQHDSSRTHGSEDS

SEQ ID NO: 108 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLAKMIHQHLSSRTHGSEDS

SEQ ID NO: 109 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLRKMIHQHLSSRTHGSEDS

SEQ ID NO: 110 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLNKMIHQHLSSRTHGSEDS

SEQ ID NO: 111 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLEKMIHQHLSSRTHGSEDS

SEQ ID NO: 112 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLGKMIHQHLSSRTHGSEDS

SEQ ID NO: 113 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLHKMIHQHLSSRTHGSEDS

SEQ ID NO: 114 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLIKMIHQHLSSRTHGSEDS

SEQ ID NO: 115 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLMKMIHQHLSSRTHGSEDS

SEQ ID NO: 116 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIHQHLSSRTHGSEDS

SEQ ID NO: 117 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLPKMIHQHLSSRTHGSEDS

SEQ ID NO: 118 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLSKMIHQHLSSRTHGSEDS

SEQ ID NO: 119 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLTKMIHQHLSSRTHGSEDS

SEQ ID NO: 120 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLWKMIHQHLSSRTHGSEDS

SEQ ID NO: 121 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLVKMIHQHLSSRTHGSEDS

SEQ ID NO: 122 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIQQHLSSRTHGSEDS

SEQ ID NO: 123 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIRQHLSSRTHGSEDS

SEQ ID NO: 124 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIVQHLSSRTHGSEDS

SEQ ID NO: 125 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIIQHLSSRTHGSEDS

SEQ ID NO: 126 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMINQHLSSRTHGSEDS

SEQ ID NO: 127 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIFQHLSSRTHGSEDS

SEQ ID NO: 128 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIYQHLSSRTHGSEDS

SEQ ID NO: 129 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIAQHLSSRTHGSEDS

SEQ ID NO: 130 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMISQHLSSRTHGSEDS

SEQ ID NO: 131 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIKQHLSSRTHGSEDS

SEQ ID NO: 132 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHYSSRTHGSEDS

SEQ ID NO: 133 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHFSSRTHGSEDS

SEQ ID NO: 134 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHSSSRTHGSEDS

SEQ ID NO: 135 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHVSSRTHGSEDS

SEQ ID NO: 136 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHNSSRTHGSEDS

SEQ ID NO: 137 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHRSSRTHGSEDS

SEQ ID NO: 138 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHISSRTHGSEDS

SEQ ID NO: 139 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHASSRTHGSEDS

SEQ ID NO: 140 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHGSSRTHGSEDS

SEQ ID NO: 141 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLQKMIHQHMSSRTHGSEDS

SEQ ID NO: 142 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLKKMIHQHLSSRTHGSEDS

SEQ ID NO: 143 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLYKMIHQHLSSRTHGSEDS

SEQ ID NO: 144 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLFKMIHQHLSSRTHGSEDS

SEQ ID NO: 145 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNQCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 146 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNVCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 147 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNICLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 148 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 149 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNYCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 150 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNACLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 151 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 152 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNQCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 153 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNVCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 154 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNICLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 155 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNECLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 156 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNYCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 157 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 158 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNPCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 159 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNQCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 160 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNVCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 161 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNICLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 162 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 163 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNYCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 164 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNACLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 165 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 166 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNQCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 167 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 168 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNICLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 169 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNECLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 170 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNYCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 171 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNACLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 172 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNPCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 173 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 174 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 175 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 176 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 177 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNQCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 178 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 179 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 180 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 181 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 182 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNVCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 183 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 184 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 185 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 186 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 187 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNICLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 188 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 189 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 190 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 191 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 192 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNECLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 193 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 194 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 195 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 196 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 197 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNYCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 198 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 199 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 200 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 201 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 202 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNACLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 203 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 204 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 205 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 206 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 207 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 208 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 209 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 210 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 211 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 212 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 213 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 214 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 215 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 216 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 217 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 218 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 219 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 220 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 221 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 222 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 223 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 224 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 225 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 226 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 227 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 228 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 229 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 230 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 231 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 232 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLHNLVIPWAPENLTLHKLSESQLELNWNNRFLNVCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 233 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNNLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 234 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNSLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 235 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNQLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 236 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 237 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLENLVIPWAPENLTLHKLSESQLELNWNNRFLNACLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 238 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 239 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLFQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 240 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLWQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 241 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLIQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 242 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYELVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 243 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYTLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 244 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 245 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYLLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 246 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 247 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFYGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 248 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFQGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 249 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFTGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 250 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGSCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 251 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGACGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 252 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGFCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 253 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGGCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 254 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCISAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 255 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 256 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNPCLEHLVQYRTDWDHSWTEQSVDYRHKFSLPSVDGQKRYTFRVRSRFNGLCYSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 257 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 258 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFNGSCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 259 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 260 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFNGLCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 261 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYGLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 262 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 263 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLFQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 264 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLWQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 265 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLIQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 266 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMELVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 267 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMTLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 268 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 269 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMLLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 270 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 271 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFYGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 272 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFQGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 273 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFTGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 274 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGSCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 275 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGACGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 276 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGFCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 277 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGGCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 278 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCISAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 279 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 280 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNPCLEHLVQYRTDWDHSWTEQSVDYRHKFSLPSVDGQKRYTFRVRSRFNGLCYSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 281 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 282 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFNGSCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 283 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFNGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 284 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFNGLCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 285 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMGLVIPWAPENLTLHKLSESQLELNWNNRFLNPCLEHLVQYRTDWDHSWTEQSVDYRHKFSLPSVDGQKRYTFRVRSRFKGLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 286 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 287 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLFQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 288 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLWQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 289 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLIQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 290 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYELVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 291 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYTLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 292 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 293 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYLLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 294 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 295 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFYALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 296 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFQALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 297 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFTALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 298 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNASCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 299 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNAACGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 300 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNAFCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 301 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNAGCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 302 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCISAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 303 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 304 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYNLVIPWAPENLTLHKLSESQLELNWNNRFLNECLEHLVQYRTDWDHSWTEQSVDYRHKFSLPSVDGQKRYTFRVRSRFNALCYSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 305 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 306 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFNASCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 307 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 308 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFNALCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 309 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLYGLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 310 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 311 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLFQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 312 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLWQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 313 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLIQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 314 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMELVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 315 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMTLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 316 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 317 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMLLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 318 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 319 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFYALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 320 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFQALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 321 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFTALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 322 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNASCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 323 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNAACGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 324 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNAFCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 325 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNAGCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 326 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCISAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 327 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 328 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMNLVIPWAPENLTLHKLSESQLELNWNNRFLNECLEHLVQYRTDWDHSWTEQSVDYRHKFSLPSVDGQKRYTFRVRSRFNALCYSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 329 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 330 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFNASCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 331 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFNALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 332 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFNALCASAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 333 - protein, 369 aa, synthetic construct
  REGION 1..369: note = Synthetic
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLLQTFVVQLQDPREPRRQATQMLKLMGLVIPWAPENLTLHKLSESQLELNWNNRFLNECLEHLVQYRTDWDHSWTEQSVDYRHKFSLPSVDGQKRYTFRVRSRFKALCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 334 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIFQHLSSRTHGSEDS

SEQ ID NO: 335 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIFQHFSSRTHGSEDS

SEQ ID NO: 336 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIFQHYSSRTHGSEDS

SEQ ID NO: 337 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIYQHLSSRTHGSEDS

SEQ ID NO: 338 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIYQHFSSRTHGSEDS

SEQ ID NO: 339 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLYKMIYQHYSSRTHGSEDS

SEQ ID NO: 340 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIFQHLSSRTHGSEDS

SEQ ID NO: 341 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIFQHFSSRTHGSEDS

SEQ ID NO: 342 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIFQHYSSRTHGSEDS

SEQ ID NO: 343 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIYQHLSSRTHGSEDS

SEQ ID NO: 344 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIYQHFSSRTHGSEDS

SEQ ID NO: 345 - protein, 209 aa, synthetic construct
  REGION 1..209: note = Synthetic
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLFKMIYQHYSSRTHGSEDS

SEQ ID NO: 346 - protein, 133 aa, synthetic construct [sequence omitted]

SEQ ID NO: 347 - protein, 519 aa, synthetic construct
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 348 - protein, 240 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 349 - protein, 538 aa, synthetic construct
MPRGWAAPLLLLLLQGGWGCPDLVCYTDYLQTVICILEMWNLHPSTLTLTWQDQYEELKDEATSCSLHRSAHNATHATYTCHMDVFHFGADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 350 - protein, 209 aa, synthetic construct
MYRMQLLSCIALSLALVTNSAPWSHPQFEKGGGSGGGSGGSSAWSHPQFEKEQKLISEEDLGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 351 - protein, 76 aa, synthetic construct
MYRMQLLSCIALSLALVTNSAPWSHPQFEKGGGSGGGSGGSSAWSHPQFEKEQKLISEEDLGGGGSGGGGSGGGGS

SEQ ID NO: 352 - protein, 209 aa, synthetic construct
MYRMQLLSCIALSLALVTNSAPWSHPQFEKGGGSGGGSGGSSAWSHPQFEKEQKLISEEDLGGGGSGGGGSGGGGSQGQDRLMIKLRQLIDIVDQLKNYVNDLVPEFLPAPEDVETNCEWSAFSCFQKAQLKSANTGNNERIINVSIKILKRKLPSTNAERRQKHRLTCPSCDSYEKKPVKEFLERFKSLLLKMIDQHLSSRTHGSEDS

SEQ ID NO: 353 - protein, 209 aa, synthetic construct
MYRMQLLSCIALSLALVTNSAPWSHPQFEKGGGSGGGSGGSSAWSHPQFEKEQKLISEEDLGGGGSGGGGSGGGGSQGQDQHMIRMRQLIDIVDQLKNYVNDLVPEFLPAPEDVETNCEWSAFSCFQKAQLKSANTGNNERIINVSIKKLKAKPPSTNAGRRQKHRLTCPSCDSYEKKPPKEFLERFKSLLQKMIHQHLSSRTHGSEDS

SEQ ID NO: 354 - protein, 538 aa, synthetic construct
MPRGWAAPLLLLLLQGGWGCPDLVCYTDYLQTVICILEMWNLHPSTLTLTWQDQYETLKDEATSCSLHRSAHNATHATYTCHMDVFHFIADEIFSVNITDQSGNYSQECGSFLLAESIKPAPPFNVTVTFSGQYNISWRSDYEDPMFYMLKGKLQYELQYRNRGDPWAVSPRRKLISVDSRSVSLLPLEFRKDSSYELQVRAGPMPGSFYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 355 - protein, 133 aa, synthetic construct [sequence omitted]